(19) United States
(12) Patent Application Publication
(10) Pub. No.: US 2004/0248083 A1
(43) Pub. Date: Dec. 9, 2004

(54) Vectors for gene therapy

(76) Inventors: Jacob Giehm Mikkelsen, Palo Alto, CA (US); Soeren Vestergaard Rasmussen, Aarhus C (DK); Mogens Duch, Risskov (DK); Finn Skou Pedersen, Aarhus (DK); Lars Aagaard, Aarhus N (DK)

Correspondence Address: FOLEY AND LARDNER, SUITE 500, 3000 K STREET NW, WASHINGTON, DC, 20007, US

(21) Appl. No.: 10/478,014

(22) PCT Filed: May 17, 2002
(86) PCT No.: PCT/DK02/00331

(30) Foreign Application Priority Data
May 17, 2001 (DK) PA2001 00789

(51) Int. Cl. C12Q 1/70; C12N 7/00; C12N 15/867

(52) U.S. Cl. 435/5; 435/456; 435/235.1

(57) ABSTRACT

The present invention relates to improved vectors useful in gene therapy which improvement particularly resides in improved safety of such vectors. The improvement is achieved by incorporating sequences into the gene therapy vector which promote dimer formation of transcripts derived from said vector. Such sequences are in a preferred embodiment self-complementary palindromic or nonpalindromic sequences.

### FIELD OF INVENTION

The present invention relates to improved vectors useful in gene therapy which improvement particularly resides in improved safety of such vectors. The present invention further relates to a process of preparing such an improved vector and its use in gene therapy.

### TECHNICAL BACKGROUND AND PRIOR ART

Many different protocols have been developed in order to cure diseases by means of introducing a therapeutic gene into a diseased organism. Many of these protocols use viral vector systems as a vehicle for introducing the desired therapeutic gene into the organism. Such vectors are e.g. based on modified adenovirus genomes or retroviral genomes. A major problem of gene therapy protocols is their safety, in particular, to avoid the production of fully functional viral genomes as the outcome of recombination events in the target or producing cells. Such recombinations might lead to fully replicative viral genomes which then could lead to uncontrolled virus replication in the recipient finally leading to cancer, as has been reported in previous reports. Many different approaches exist in order to improve the safety of virus based gene therapy vectors which all have in common that the occurrence of replication competent viruses in the organism to be treated shall be minimised. Most of these approaches make use of non-functional viral genomes which do not contain all information required for virus replication or packaging. In one such approach, the primer binding site (PBS) of a retroviral vector has been modified to a sequence that does not allow strong base pairing with the tRNA primer. Such a modified retrovirus should be incapable of replication because an essential element to start viral replication, the primer binding site, is no longer functional. What is required, however, is a producer or packaging cell which is capable of producing functional viral particles. Such producer cells generally do contain the means which can complement the deficiency of the viral genome intended for introduction into the patient. Said producer cells bear the risk that by recombination events within the producer cells not only non-functional virus genome is packaged into virus particles but also genomes which, by recombination events, become fully replicative.

A widely used vector system for use in gene therapy protocols are retroviral vectors which are gene transfer vehicles derived from birds and mammals that exploit features of the retrovirus replication such as high infection efficiency and stable co-linear integration of the virally transmitted information in a target cell chromosome. Retroviral vectors are becoming important tools in basic research, biotechnology and gene therapy.

Most retroviral vectors currently in use are derived from murine leukaemia viruses (MLVs). MLVs are particularly suitable as vectors due to their well-documented pattern of transcription in diverse cell types and relatively simple modular genetic structure. The retroviral structure, for example, is disclosed in detail in U.S. Pat. No. 6,037,172.

Retroviruses replicate through a double-stranded DNA intermediate which is stably integrated into the host genome as proviral DNA. Upon infection of germ cells, proviruses are transmitted through the germ line and may persist as genetic entities and Mendelian genes in the genome for multiple generations. Such elements of the genome, usually referred to as endogenous retroviruses (ERVs), are often replication-defective due to the accumulation of mutations and, in order to spread, rely on concomitant replication of helper viruses. ERV-derived RNA may thus hitchhike with virus particles released from the host cell, provided that structural cis-elements within the ERV packaging signal facilitate recognition by the helper virus RNA encapsidation machinery. Among a panel of known murine ERVs, members of the murine leukaemia virus (MLV)-related and VL30 ERV families are selectively included in MLV virions.

Encapsidation of two genomic RNA molecules into budding retrovirus particles allows for recombination during reverse transcription within the viral core particle internalised in the cytoplasm of the infected cell. Retrovirus recombination occurs primarily by template switching of nascent minus-strand DNA. Coexistence and occasional copackaging of retroviral RNAs of exogenous and endogenous origin allow for the generation of recombinant proviruses harbouring sequence of both parental originals. In such a scenario, events of template switching during DNA synthesis may facilitate recombinational patch repair of virus mutations by substituting defective segments of the genome with functional sequence patches provided by the copackaged endogenous virus. Previously reported examples of ERV-based recombinational reversion include patch repair of virus mutants harbouring (i) modification of the integrase attachment site (ii) deletions within IN and PR regions of the pol gene; and (iii) defective primer binding sites (PBS; Mikkelsen et al., J. Virol. 70: 1439-1447, 1996 and 3. Virol. 72: 6967-6978, 1998). In addition, recombination events including both packaging construct and ERV-derived RNA have been found to result in “patch repair” of retrovirus vectors, leading to the hazardous generation of replication-competent viruses in retrovirus-based gene transfer applications.

RNA dimerization and encapsidation are tightly coupled events of retrovirus replication. Whereas copackaging of retroviral RNAs is a proven prerequisite for recombination, there remains an open question of whether RNA dimer formation is required for packaging and/or genetic interactions between copackaged RNAs. For all retroviruses studied, the primary determinants of RNA dimerization and encapsidation have been mapped to the packaging signal within the 5′ untranslated region (5′ UTR) situated downstream from the PBS and upstream from the gag initiation codon. A specific stem-loop structure in the 5′ UTR facilitates synthetic RNA dimer formation in vitro through intermolecular “kissing” of conserved palindromic loop motifs (Clever et al., 1996; Fosse et al., Biochemistry 35: 16601-16609, 1996; Girard et al., Biochemistry 34: 9785-9794, 1995; Haddrick et al., J. Mol. Biol. 259: 58-68, 1996; Laughrea & Jette, Biochemistry 33: 13464-13474, 1994; Paillart et al., Proc. Natl. Acad. Sci. USA 93: Biochemistry 35: 16601-16609, 1996, 1997). Within a 5′ UTR recombination window, this kissing-loop sequence is a hotspot for nascent minus-strand DNA template switching between vector donor RNA and endogenous virus-derived acceptor RNA templates (Mikkelsen et al., 1996, 1998b). The influence of mutations in the kissing-loop sequence on infectivity and template shift has been analysed by Mikkelsen et al. (2000) 3. Virol. 74, 600-610.

Further information on recombination events in retroviruses is also given in Mikkelsen and Pedersen (2000) J Biomedical Sci. no. 7, pages 77-99.

While a wide number of strategies have been attempted to improve the safe retroviral vectors for gene therapy the problem of unwanted recombination between the retroviral vector and endogeneous or contaminating exogenous virus largely seem unsolved. Although extremely rare such recombination events may lead to the formation of functional virus genome which is packaged into fully replicative virus particles. To fully exploit the potential of retroviral vectors as carriers of therapeutically important genes the harmful effects of such unwanted recombination events must be sought minimised by minimising the likelihood of such recombination's. The present invention discloses a method and the means for minimising the frequency of unwanted recombination.

### SUMMARY OF THE INVENTION

An object underlying the present invention was to provide a vector useful in gene therapy protocols and having improved safety and a process for preparing such vectors.

Said object is achieved by a retroviral based vector system which contains at least one modified heterologous or synthetic dimerisation sequence which promotes dimer formation of transcripts derived therefrom and which results in a reduction of the recombination frequency between a transcript of the vector and those of a different retrovirus present in a cell.

The term “foreign sequence that promotes dimerisation” or the term “modified heterologous or synthetic dimerisation sequence” are herein used interchangeably. A “modified heterologous or synthetic dimerisation sequence” means in this context that the sequence in question whether it is derived from a biological source or whether it is a artificial sequence is not found at the same position in the wild type nucleic acid molecule which was used to construct the vector system according to the invention, and that the sequence is a dimerisation sequence. For example, if the region between the 5′ UTR and the gene of interest is modified by incorporation of a sequence such incorporated sequence is a “modified heterologous or synthetic dimerisation sequence” even if it is derived from the same viral genome but from a different region. However typically the “modified heterologous or synthetic dimerisation sequence” is obtained from a source which is different from the wild type nucleic acid molecule which was used to construct the vector system according to the invention. Any such “modified heterologous or synthetic dimerisation” sequences can easily be detected by sequence comparison of the non-modified starting retroviral genome and the genome modified according to the invention.

In the present context the term “different retrovirus” define a retrovirus which contains a genome that is different from the at least one retrovirus-genome coded transcript of the specific retroviral based vector system. DETAILED DISCLOSURE OF THE INVENTION

As has been explained above, the lifecycle of a retrovirus comprises a dimer forming step during virus replication. Moreover, as also has been explained above, the generation and replication of viral vectors for use in gene therapy protocols requires specific producer cells which provide the machinery for producing complete viral particles because the retrovirus derived vector carrying the gene of interest is deficient in essential elements for virus replication and/or packaging. The problem that may arise in a packaging cell is that recombination events between the gene therapy vector and endogenous sequences of the packaging cells and/or copackaging of the gene therapy vector with endogenous sequences of the packaging cell may lead to the production of viral particles which contain functional viral genomes which when entering the target cell in the patient to be treated by gene therapy and replicating therein leading to harmful results in the patient (see e.g. FIG. 1).

Therefore, the concept underlying the present invention is based on the findings that the transcripts being produced from the vector system according to the invention show a high affinity towards each other which means that it is much more likely that dimers are formed being derived from transcripts of the gene therapy vector introduced into the packaging cell versus dimers comprising transcripts of endogenous virus genomes.

The sequence which is introduced into the gene therapy vector in order to promote the dimer formation can basically be inserted at any part of the vector which is transcribed into RNA.

Preferably the sequences, however, are inserted at a region around the 5′ UTR region of the retrovirus genome preferably between the 5′ UTR region and the gene of interest to be introduced into the patient, see e.g. FIG. 4 and examples 4 and 6, wherein RNA II and RNA I are the sequences which promote dimer formation.

In a preferred embodiment, the vector system is a single vector, e.g. a conventional retroviral vector being used in gene therapy which comprises a stretch of nucleotides of heterologous, preferably of nonviral, origin and which contains sequence element(s) selected from self-complementary palindromic or nonpalindromic sequences. Such a type of sequence allows the hybridisation between two transcripts from said single vector which hybridisation then promotes the dimer formation of transcripts. Such an approach is shown e.g. in FIG. 7. One vector according to the invention is a retroviral vector, shown in the FIG. 7, which contains the therapeutic gene of interest and a synthetic RNA motif which promotes, by high self-affinity of the synthetic sequence, homodimer formation. This in turn favours the formation of dimers between the transcripts derived from the vector according to the invention over endogenous transcripts, the latter not being desired due to the risk that by recombination a complete and functional retroviral genome is created. The promotion of the homodimer formation by the synthetic RNA results in a high degree of encapsulation of homodimeric vector RNA, which RNA cannot replicate in the target cell but is capable of introducing a therapeutic gene of interest into the target cell genome.

According to a further preferred embodiment, a homodimer is formed between transcripts which are derived from one gene therapy vector only.

In a further preferred embodiment the vector systems according to the invention comprise a first and a second vector. Such a two-vector-system is shown in FIG. 2b wherein the vector system according to the invention comprises a rescue vector and a retroviral vector which comprises the therapeutic gene of interest. In this system the first vector, e.g. the retroviral vector, contains a first sequence element and the second vector, e.g. the rescue vector contains a second sequence element, which first and second sequence elements how high affinity to each other and promote the heterodimer formation between the transcripts derived from the retroviral vector and the rescue vector. Again, also in this system, the dimer formation between the first and second vector is promoted versus other possibly competing heterodimers, such as dimers formed between transcripts of the retroviral vector according to the invention and endogenous packaging cell derived transcripts, the latter being non-desired for packaging into viral particles. In said two-vector-embodiment it is preferred that a heterodimer is formed between a transcript of the first vector, e.g. the retroviral vector with the therapeutic gene of interest and the second vector, e.g., the rescue vector. The rescue vector mainly serves as a source for the further transcript to be packaged along with the transcript carrying the therapeutic gene of interest.

In the two-vector-systems according to the invention, the dimer formation can be promoted by any pair of sequences which show high affinity to each other. Such sequences can be selected from complementary sequences occurring in nature, such as the ColE1RNAI and ColE1RNAII sequences, sequences from the so-called “kissing loop” of retrovirus or synthetic sequences which are designed such that the first sequence being introduced into the first vector shows strong hybridisation with the second sequence inserted into the second vector. As to whether a given synthetic sequence or pair of sequences is capable of promoting dimer formation can be tested, as illustrated in the examples and shown in FIG. 8 which will be explained in more detail below.

In a particularly preferred vector system according to the invention, the vector(s) is (are) based on retroviral vectors which are already well-established in the art (see e.g. U.S. Pat. No. 6,037,172).

In a further preferred embodiment, a vector system according to the invention is useful for introducing at least one therapeutic gene of interest into a recipient. The gene of interest can be e.g. a gene which should be replaced or supplemented in the recipient. Alternatively, the therapeutic gene is a sequence which is suitable for blocking and/or reducing the gene activity in a host. Moreover, the therapeutic gene can also encode for a product which shall effect biological responses such as the immune response. The gene therefore encodes e.g. an antigen against which an immune response of the recipient is desired.

The transcript according to the invention can be any conventional transcript of retroviral vectors which transcript, however, contains the additional sequence which promotes the dimer formation of the transcript during its replication in the producing cell. A preferred form of the transcript is a homodimer form thereof as it is produced in a packaging cell. In a further preferred embodiment two different transcripts are produced from a first and a second vector according to the invention, the respective transcripts containing sequence elements which promote the heterodimer formation of said two transcripts.

A further embodiment of the invention concerns a packaging cell for packaging transcripts derived from gene therapy vectors according to the present invention. Useful packaging cells for receiving the vector system according to the invention can be selected from ψ2 (Mann et al., 1983, Cell 33:153-159), BOSC23 (Pear et al., 1993, PNAS 90:8392-8396), PA317 (Miller & Buttimore, 1986, Mol. Cell. Biol. 6:2895-2902), GP+envAm12 (Markowitz et al., 1988, Virology 167:400-406), PG13 (Miller et al., 1991, 3. Virol. 65:2220-2224), and related packaging cell lines.

The present invention moreover relates to new viral particles which contain a transcript produced from the vector system according to the invention. A preferred virus particle contains a homodimer of a transcript which is derived from a single vector. Such viral particles then can infect a target cell and incorporate into the target cell the genome of a retroviral vector according to the invention. The target cell being infected by the virus particle according to the invention incorporates into its genome the therapeutic gene of interest derived from the virus particle but is not capable of replicating the viral genome due to lack of essential elements required for replication and/or packaging.

The present invention further relates to a pharmaceutical formulation or a vaccine which comprises a vector system or viral particle according to the invention. Such compositions may contain the conventional additives required for stabilisation and/or administration of the vector or viral particle.

The present invention also relates to a cell having incorporated in its genome a sequence which is derived from the vector system according to the invention.

The invention further relates to a process for preparing the vector system according to the invention. According to said process, a conventional retroviral vector is modified by inserting at least one sequence which promotes dimer formation of the transcript being derived from said vector. The sequence can, e.g. be synthesised or be isolated from a naturally occurring useful sequence. The introduction of such a sequence into a given vector system is within the skills of the skilled person.

Preferred sequences to be used in the process according to the invention are selected from self-complementary sequences including (i) palindromic and synthetic nonpalindromic sequences of retrovirus kissing loops, (ii) sequences of plasmid origin: ColE1 (RNA I and RNA II); IncF (cop A RNA and repA mRNA); IncI (inc RNA and repZ mRNA); ColE2 (copRNA and repmRNA); R1162 (ct RNA and rep1 mRNA); R6K (silencer and activator); pT181 (RNA I and repC mRNA); IncF (finP RNA and traJ mRNA); IncFII (sok RNA and hok mRNA). (iii) CopA/CopT-derived sequences involved in plasmid replication in bacteria, (iv) Sequences of phage origin: lambda (aQ RNA and Q mRNA), lambda (oop RNA and cII mRNA); P22 (sar RNA and ant mRNA); P1, P7 (c4 repressor and ant mRNA), (v) sequences of transposon origin: IS10 (RNA-OUT and tnp mRNA), and (vi) sequences of bacterial origin: E. coli (micF RNA and ompF mRNA); E. coli (tic RNA and crp mRNA) and the bacterial finOP antisense RNA recognition system involved in translation regulation. Moreover, preferred sequences include (vii) complementary nonviral or synthetic sequences known to facilitate efficient antisense recognition.

Basically, the sequence can be inserted at any position in the retrovirus as long as said position is transcribed by the machinery of the producer cell wherein the vector is inserted for virus production.

Preferably, the sequence is inserted between the 5′ UTR and the 5′ site of a gene of interest contained in the gene therapy vector.

What is achieved by said process is that the obtained vector is more safe in gene therapy protocols, particularly in that the risk of packaging of undesired genomes into viral particles by the producer cell is reduced.

The present invention further relates to a method for determining the capability of a sequence to promote homodimer and/or heterodimer formation of transcripts derived from a retroviral gene therapy vector which method comprises the step of introducing a vector system containing such sequence into a producer cell and measuring the rate of homodimer and/or heterodimer formation of transcripts derived from said vector system. The in vivo set-up of one such a method is shown in FIGS. 8a and 8b for a dual vector system. In more detail, in the example shown, a first vector comprising the sequence element RNAII and a functional primer binding site (PBS) but lacking the R-region, and a second vector containing the sequence element RNAI, but having a non-functional primer binding site (PBSX3) but containing the R-region are introduced into the cells. RNAII and RNAI is an example of a sequence pair which promotes dimer formation between the transcript of the first vector and the second vector. In this system, a complete, functional transcript is produced only if the heterodimer as shown is formed. The first vector is not capable of producing a functional transcript because it lacks the R-region. This region is, however, required as a template for the tRNA having attached thereto the US and R sequence of the 5′ end of the first vector. If this partial reverse transcript shall be completed, this can be accomplished only by a strand switch to the 3′ end of the second vector since only the second vector contains the R-region which can be used as a template. Such a strand switch can, however, occur only if a heterodimer is formed as illustrated in FIG. 2a. It is to be noted that also the second vector cannot be replicated because of the non-functional PBS site. The result of this heterodimer formation and the reverse transcription thereof is that a transcript is formed which contains as a marker gene the neomycin resistance gene (Neo) which confers resistance to G418 on eucaryotic cells (FIG. 2a) or the yellow fluorescent protein (YFP) on FIGS. 8a and b, which gene marks those cells having incorporated therein the respective retrotranscript. Therefore, a high number of G418 resistant or yellow stained cells indicates an efficient heterodimer formation which, hence, is an indication for a useful sequence promoting such dimer formation. In the absence of such a sequence, RNAII in the first vector and RNAI in the second vector, one would expect a very low rate of yellow-stained cells due to insufficient or poor heterodimer formation and, hence, a very low rate of retrotranscript product containing the YFP gene.

A similar approach can also be used for a single vector system wherein a sequence is measured with respect to its homodimer formation capability. In such a system DNA is packaged only if it efficiently forms a homodimer in the producer cell. Again, in such a system a synthetic sequence sufficiently promotes homodimer formation which results in a high frequency of packaging of the homodimer, which in turn leads to a high rate of cells carrying in its genome the respective transcript containing, e.g. a marker gene such as the YFP. Obtaining yellow stained cells again indicates that the sequence tested was useful to promote homodimer formation for packaging.

The present invention further relates to the use of the vector system according to the invention for preparing a composition which is useful in gene therapy. The vector system according to the invention can be administered to the organism to be treated as a pharmaceutical formulation or as a vaccine. The skilled person is well aware of the large number of different protocols for introducing a heterologous or synthetic DNA into a host organism for the purpose of gene therapy. Alternatively, viral particles as produced by a producer cell can be administered to the organism to be treated.

The present invention relates to a retrovirus based vector system useful for gene therapy, characterized in that it contains a foreign sequence that promotes homodimer or heterodimer formation of transcripts derived therefrom. In particular the invention relates to a retroviral vector system comprising at least one retroviral vector which has at least one modified heterologous or synthetic dimerisation sequence not present in its wild type state, said modification resulting in a reduction of the recombination frequency between a transcript of said vector that includes a transcript of the sequence modification, and at least one transcript of at least one different retrovirus present in a cell wherein the vector is present, said reduction being at least 2-fold relative to that of a corresponding transcript from the wild type retroviral vector. In the present context the reduction of the recombination frequency between a transcript of the vector system and at least one transcript of a different retrovirus present in a cell is determined as described in example 2 for Akv-MLV-derived vectors modified in the kissing-loop region and a simulated endogenous virus.

In a preferred embodiment thereof, the vector system is a single vector comprising a stretch of nucleotides which contains a sequence element(s) selected from self-complementary sequences known to facilitate RNA-RNA recognition in a nonviral context. In particular the invention relates to a system in which the vector, relative to the wild type vector from which it is derived, has a transduction titer which is at least 25%.

In a further preferred embodiment the vector system produces transcripts that form a homodimer in a producing cell.

In a further preferred embodiment the vector system comprises a first and second vector, the first vector lacking an initiation site for reverse transcription that is present in the second vector.

In a further preferred embodiment the first vector comprises a sequence which is complementary to the sequence of the second vector.

In a further preferred embodiment the two vector system produces transcripts that form a heterodimer in a producing cell which dimer is composed of a transcript of the first vector and a transcript of the second vector. To accomplish the directed formation of hetrodimers of the two vectors one embodiment of the invention is a system comprising a first and a second retroviral vector, the first vector lacking an initiation site for reverse transcription that is present in the second vector.

In a further preferred embodiment the complimentary sequence which shall promote dimer formation is selected from (i) palindromic and synthetic nonpalindromic sequences of retrovirus kissing loops, (ii) sequences of plasmid origin: ColE1 (RNA I and RNA II); IncF (cop A RNA and repA mRNA); IncI (Inc RNA and repZ mRNA); ColE2 (copRNA and repmRNA); R1162 (ct RNA and rep1 mRNA); R6K (silencer and activator); pT181 (RNA I and repC mRNA); IncF (finP RNA and traJ mRNA); IncFII (sok RNA and hok mRNA). (iii) CopA/CopT-derived sequences involved in plasmid replication in bacteria, (iv) Sequences of phage origin: lambda (aQ RNA and Q mRNA), lambda (oop RNA and cII mRNA); P22 (sar RNA and ant mRNA); P1, P7 (c4 repressor and ant mRNA), (v) sequences of transposon origin: IS10 (RNA-OUT and tnp mRNA), and (vi) sequences of bacterial origin: E. coli (micF RNA and ompF mRNA); E. coli (tic RNA and crp mRNA) and the bacterial finOP antisense RNA recognition system involved in translation regulation. Moreover, preferred sequences include (vii) complementary nonviral or synthetic sequences known to facilitate efficient antisense recognition.

In the embodiment which comprises a first and a second vector the resulting transduction titer is to a large extent depending on the ratio in which the two vectors are present in a cell. In particular the present invention relates to an embodiment in which the two vectors, when present in a cell in a ratio in the range of 1:9 to 9:1, have a transduction titer which is at least 1% relative to the transduction titer of the wild type vector(s) from which they are derived.

In a further preferred embodiment the vector system comprises at least one therapeutic gene of interest which therapeutic gene is used for curing and/or preventing a disease in a recipient to be treated; or for immunising the recipient.

The present invention further relates to a transcript derived from a vector system according to the invention

in a preferred embodiment the transcript is capable of forming a homodimer in a packaging cell;

in a further preferred embodiment the transcript is capable of forming a heterodimer in a packaging cell.

The present invention further relates to new viral particles which are characterized in that they contain a transcript derived from a vector system according to the invention.

The present invention further relates to a packaging cell for packaging a gene therapy vector which packaging cell is characterized in that it contains a vector system according to the invention. In yet an embodiment the invention relates to a packaging cell for replication of at least one retroviral transfer vector of a system is a mammalian or avian cell which has been transformed by the insertion of one or more DNA sequences carrying the information for the production of viral proteins required in trans for replication of said at least one retroviral transfer vector.

The present invention further relates to a process for preparing a vector system useful for gene therapy comprising the step of incorporating at least one sequence into at least one vector useful in gene therapy which sequence promotes dimer formation of transcripts derived therefrom. In yet an embodiment of the invention the process for preparing a retroviral vector system, comprising the step of introducing at least one sequence modification in a dimerisation sequence, said modification resulting in a reduction of the recombination frequency between a transcript of said vector that includes a transcript of the sequence modification, and at least one transcript of at least one different retrovirus present in a cell wherein the vector is present, said reduction being at least 2-fold relative to that of a corresponding transcript from the wild type retroviral vector.

In a preferred embodiment the dimerisation sequence is selected from a self-complimentary sequence of nonviral origin (ColE1RNAI and ColE1RNAII, CopA/CopT, finOP, nonpalindromic kissing-loop sequences, synthetic antisense systems, etc.), however according to the present invention other dimerisation sequences are contemplated. Yet an aspect the present invention provides a method for determining the capability of a sequence pair comprising two identical or two different sequences to promote homodimer and/or heterodimer formation of transcripts derived from a retroviral vector system, the method comprising the step of introducing at least one retroviral vector of said vector system into a host cell and evaluating the frequency of homodimer and/or heterodimer formation of transcripts derived from said vector system by quantitating the relative number of specific recombination events that have occured, said number of specific recombination events being obtained by a method comprising the following steps;

a) selecting said sequence pair.

b) inserting one member of said sequence pair into one retrovirus vector containing a selectable marker gene and a non-functional primer binding site (PBS), and inserting the other member of said sequence pair into another retrovirus vector containing a functional primer binding site (PBS) but not containing the same selectable marker gene as the previous vector,

c) co-introducing the two retrovirus vectors of step (b) into a suitable pagaging cell which provides the necessary means to allow formation of infective retrovirus particles containing the information from both of said two retrovirus vectors of step (b),

d) recovering virus containing media and infect a culture of suitable host cells which do not contain the selectable marker gene of the said one retrovirus vector containing a non-functional primer binding site of step (b),

e) subjecting the transfected host cells of step (d) to a selection procedure which only allow cells that have been infected with virus particles containing said selectable marker gene of step (d) to form colonies, and

f) quantitating the number of specific recombination events from the number of resistant colonies obtained.

In a further preferred embodiment the process is applied to prepare a vector system useful for gene therapy and the sequence which promotes dimer formation is incorporated in a region supposed to provide optimal functionality preferably in a region between the 5′ UTR and the leader sequence of a therapeutic gene of interest.

The present invention further relates to a method for improving the safety of gene therapy vector systems comprising the step of introducing into a vector system useful for gene therapy at least one sequence into at least one vector useful in gene therapy which sequence according to the present invention results in a reduction of the recombination frequency between a transcript of said vector that includes a transcript of the sequence modification, and at least one transcript of at least one different retrovirus.

The present invention further relates to a method for determining the capability of a sequence to promote homodimer and/or heterodimer formation of transcripts derived from a gene therapy vector which method comprises introducing a vector system containing such a sequence into a host cell and measuring the rate of homodimer and/or heterodimer formation of transcripts derived from said vector system.

The present invention further relates to the use of a vector system according to the invention or a viral particle according to the invention for preparing a composition useful in gene therapy.

The present invention further relates to a pharmaceutical formulation or a vaccine comprising a vector system according to the invention and/or a viral particle according to the invention.

The present invention further relates to a target cell having being infected with a vector system or a vector particle according to the invention.

### BRIEF DESCRIPTION OF FIGURES

FIG. 1. Shows the generation of replication competent virus from packaging cells used in gene therapy.

FIG. 2a. Shows an in vivo two-vector forced recombination system that promotes heterodimerization and reduces recombination with endogenous virus transcripts.

FIG. 2b. Shows schematically the template shift during the reverse transcription of the RNA dimer, giving rise to the recombination event.

FIG. 2c. Shows the retroviral vectors used in the set-up introducing non-palindromic sequences into the kissing loop region to direct heterodimerization.

FIG. 3. Shows examples of retroviral vectors comprising heterologous sequences and in vivo determination of the effect of insertion as measured by transduction efficiencies.

FIG. 4. Shows the outline for the assay to determine the ability of RNA elements to facilitate heterodimerization of RNA.

FIG. 5. Shows the oligo-tagging assay for testing the potetential of any two sequences to form heterodimers in virions, for details se Example 10.

FIG. 6. Shows a schematic model for immobilization of retroviral vectors facilitated by heterodimerization though pairs of matching non-palindromic sequences.

FIG. 7. Shows the single vector system according to the invention, wherein a single vector is modified by inserting a homodimer forming dimerization sequence.

FIGS. 8a and 8b. Shows an in vivo set-up for testing the homodimer and heterodimer forming capabilities of a sequence useful in a gene therapy vector.

### EXAMPLES

The following examples further illustrate the invention.

### Example 1

### Generation of Replication-Competent Virus from Packaging Cells Used in Gene Therapy

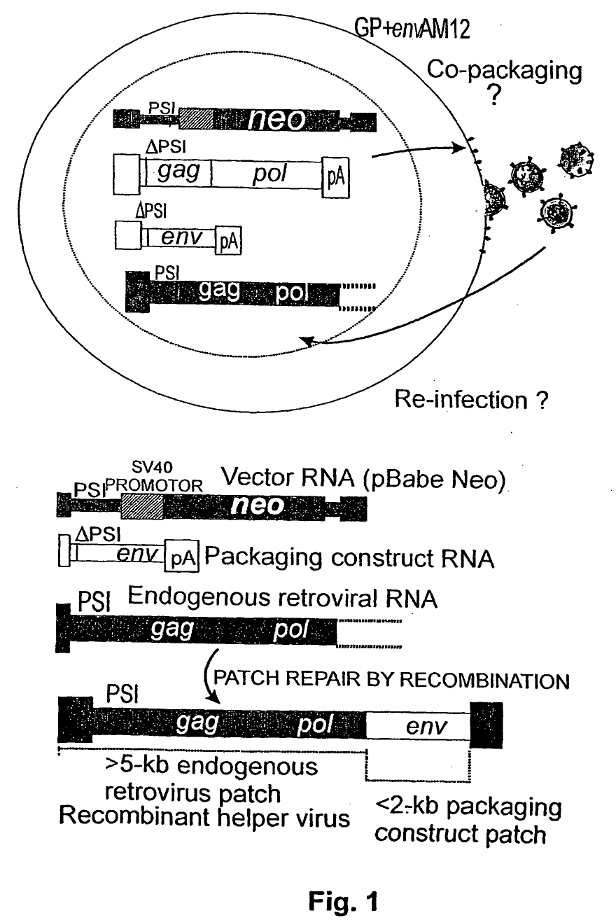

In a conventional protocol for producing virus particles for use in gene therapy, a producer cell providing the machinery for replicating and packaging a retroviral genome is used (see FIG. 1). In the example shown, the packaging cell contains endogenous retroviral RNA containing the gag and pol gene.

As a packaging construct, a construct is used which contains the env gene. The vector which is designed for introduction into the target cell contains in the present case the SV 40 promoter and the neo gene. None of such constructs is per se capable of replication and packaging. However, if recombination between the packaging constructs RNA and the endogenous retroviral RNA occurs, a fully functional recombinant virus is created which can then be harmful to the recipient due to its uncontrolled replication and growth in the recipient.

### Example 2

### The Presence of an Alternative Palindrome in Akv-Based Vectors Reduces Recombination

experiments showing that Akv-MLV-derived vectors modified in the kissing-loop region by insertion of an alternative palindrome can reduce recombination with a simulated endogenous virus

Introduction:

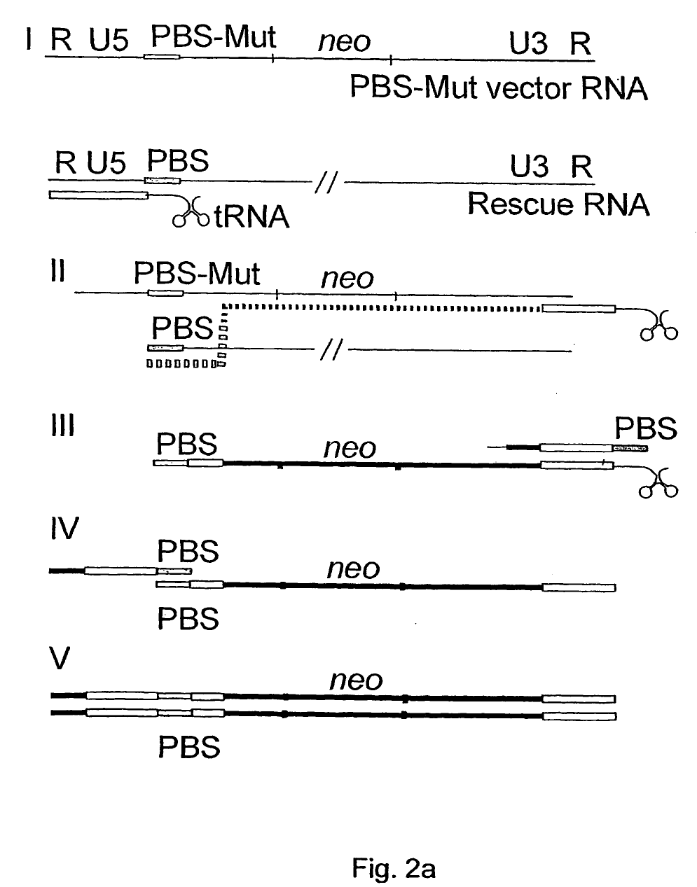

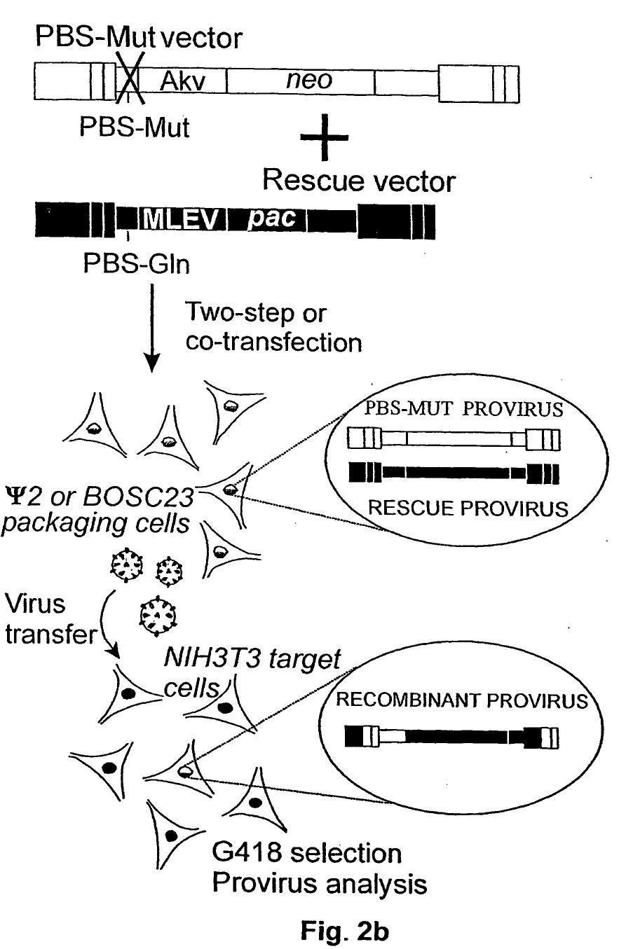

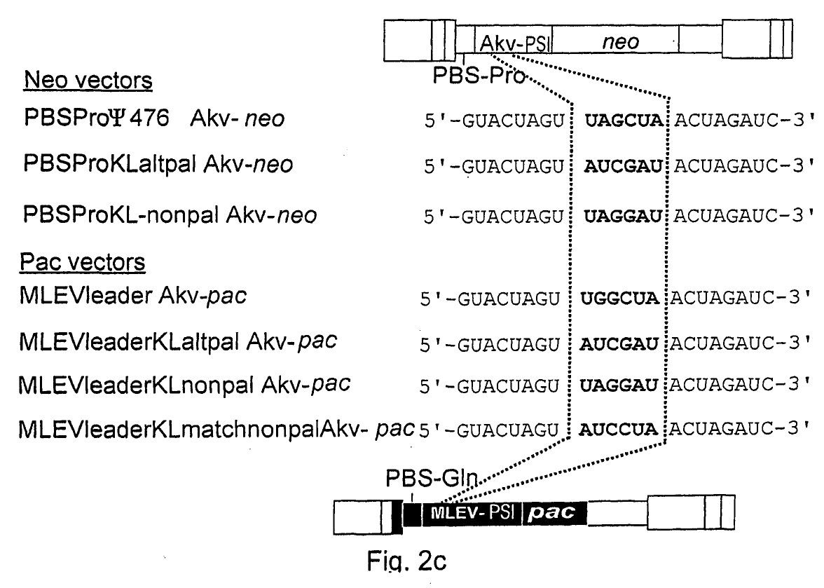

In the in vivo situation a vector RNA may co-package with an endogenous virus transcript and through recombination during the process of reverse transcription, the endogenous virus may donate functional sequences. Eventually, this may lead to the generation of replication competent viruses (see example 1). In murine cells (Psi2 cell line) PBS mutated Akv-derived vectors have been shown to give rise to rare recombination events with an endogenous murine leukemia virus (MLEV) (Mikkelsen et al., J. Virol. 70: 1439-47, 1996) probably due to low expression of endogenous virus. In order to perform a quantitative study and additionally to modify sequences in both vector and endogenous virus a human cell line system was established. The leader of the endogenous virus was cloned into a vector construct and a human packaging cell line was transfected with the MLEV-derived vector and the Akv-derived vector simultaneously. Consequently, expressing a vector construct harboring the endogenous virus sequence mimicked the expression of such endogenous virus. Additionally, site specific mutations could be introduced in both the Akv vector and the MLEV-derived vector. As no endogenous viruses have been found to recombine with Akv-derived vectors in human cell lines background, recombination with naturally occurring endogenous virus was eliminated. The experiments were carried out in a human packaging cell line (BOSC23; Pear et al., Proc. Natl. Acad. Sci. U.S.A. 90: 8392-6, 1993) as a two-vector forced recombination system to study the potential for template switching during the process of reverse transcription as described below and illustrated in FIG. 2a.

One vector harbored a mutated non-functional PBS (PBSmut) (e.g. pPBSMut476 Psi Akv-neo) which is co-transfected together with a vector containing a wildtype PBS (e.g. PMLEVleader Akv-pac). Heterodimers may form between the two RNA transcripts resulting from the two vectors and may co-package in a virus particle. During reverse transcription 1'st strand synthesis initiates from the strand with an intact PBS. As the vector constructs comprise homologous R regions, the first strand transfer results in transfer to each strand. In case of an interstrand transfer (strand transfer to the other strand) the 1'st strand synthesis would proceed through the selection gene. However, due to the need for complementarity between the individual PBS regions for second strand transfer, a template shift (recombination) has to occur at some point during first strand synthesis. As the target cells are selected for resistance conferred by the gene contained with the PBS mutated construct recombination is directed to the leader region outside the coding region, see FIG. 2b. The vector harboring the intact PBS is consequently referred to as the rescue vector as this vector may complement the PBS mutated vector.

Due to a “background” level of rescue probably arising through initiation of first strand synthesis from other regions than the PBS, transduction of target cells may take place even in the lack of rescue vector. However, including the rescue vector strongly increases the transduction efficiency. The magnitude of the increase in rescue titer depends on how efficiently the two vectors recombine which is probably strongly influenced by the vector RNAs ability to heterodimerize. Several experiments indicate that dimerization is a prerequisite for packaging thus heterodimerization is a prerequisite for recombination (Hu and Temin, Proc. Natl. Acad. Sci. U.S.A. 87: 1556-1560, 1990).

Description of Vector Constructs:

A panel of transfer vectors harboring modifications of the kissing-loop dimerization sequence was derived from an Akv MLV-based retroviral vector harboring the wildtype Akv-MLV 5′ leader region including the 476-bp packaging region, Psi (the region intervening U5/PBS and leader/gag junctions). This vector, which in its plasmid form is designated pPBSPro476Psi Akv-neo, contains the Akv-MLV long terminal repeats, a primer-binding site (PBS) matching proline tRNA, and the neomycin resistance gene (neo) flanked upstream by the 5′ leader region and downstream by 480-bp Akv-MLV sequences including the 3′ untranslated region, see FIG. 2c.

In brief, PBS knockout vectors harboring neo and rescue vectors harboring the puromycin resistance gene (pac) were generated as follows: the pac gene was PCR-amplified from pPUR (Clontech Laboratories, Inc.) with primers: Fw Pac ampli: 5′-ATCGGGGGATCCCTTCCATGACCGAGTACAAGCCC-3′ (SEQ ID NO: 1), Rev Pac ampli: (5′-TATCCAGATGAACAGCATTCGCGGGTCGTGGGGCGGGCGT-3′ (SEQ ID NO: 2)), containing BamHI and BsmI restriction sites, respectively. The amplified 634-bp fragment was inserted by standard cloning procedures into BamHI-BsmI-digested (restriction sites flanking neo) pPBSPro476Psi Akv-neo and pPBSMut476Psi Akv-neo, the latter containing a defective primer-binding site as previously described (PBS-Umu in Mikkelsen et al., J Virol 72: 6967-78, 1998 generating pPBSPro476PsiAkv-pac and pPBSMut47PsiAkv-pac.

The 465-bp packaging region of MLEV, a previously described MLV-like endogenous virus (GenBank accession no. AF041383; Miele et al., J Virol 70: 944-51, 1996), was PCR-amplified from pPBSGln465MLEVPsiAkv-neo harboring a functional glutamine PBS as part of the original MLEV leader sequence (Mikkelsen et al., J Virol 74: 600-10, 2000), using primers: Fw MLEV ampli: 5′-AGATTGATTGACTGCCCACCTCGGGGGTCTTTCATTTGG-3′ (SEQ ID NO: 3), and Rev MLEV ampli: 5′-GGGCGCCCCTGCGCTGACAGCCGGAACAC-3′ (SEQ ID NO: 4). The MLEV-Psi fragment was connected by overlap extension and PCR with a fragment containing the Akv LTR. The Akv LTR PCR fragment derived from a PCR using primers: Akv U5 fw: 5′-GGGAATTCTACCTTACGTTTCCCCGACCAGAGCTGATGTTCTCAG-3′ (SEQ ID NO: 5), and Akv rev: 5′-GGTGGGCAGTCAATCAATCTGAGGAGAC-3′ (SEQ ID NO: 6) and the overlap reaction using primers: Overlap fw: 5′-GGGAATTCTACCTTACGTTT-3′ (SEQ ID NO: 7) and Overlap rev: 5′-CAGGTCGACGGATCCGATCTCGAAAACACTTAGAC-3′ (SEQ ID NO: 11). The overlap extension reaction resulting in an Akv-MLEV chimeric fragment (the Akv-MLEV junction being in the U5 region) that was cloned into the appropriate position of pPBSPro476 Psi Akv-pac, generating pMLEVleaderAkv-pac, see FIG. 2c.

The alternative palindromic loop motif (KL-altpal for kissing loop alternative palindrome) was introduced into the kissing-loop sequences of pPBSPro476PsiAkv-neo, pPBSMut476PsiAkv-neo, and pMLEVleaderAkv-pac (yielding pPBSPro476PsiAkv-neo KL-altpal, pPBSMut476PsiAkv-neo KL-altpal, pMLEVleaderAkv-pac KL-altpal) by PCR-mediated mutagenesis using the following sense oligonucleotides matching Akv, or corresponding MLEV, positions 291 to 330 (Van Beveren et al., In “RNA tumor viruses” [N. Weiss, H. Teich, H. Varmus, and J. M. Coffin, Eds.], Vol. 2. Cold Spring Harbor Press, Cold Spring Harbor, N.Y., 1985) (the altered loop sequence is underlined): ON1 (5′-CTGATTCTGTACTAGTATCGATACTAGATCTGTATCTGGC-3′ (SEQ ID NO: 8)) introducing the alternative palindrome 5′-ATCGAT-3′ (SEQ ID NO: 9) (KL-altpal). Together with an antisense oligonucleotide matching the most 3′ part (positions 617 to 638) of either Akv-Psi (ON8, 5′-CAGGTCGACGGATCCGTTTTTAGAAGCGGTCCAMAC-3′ (SEQ ID NO: 10)) or MLEV-Psi (ON9, 5′-CAGGTCGACGGATCCGATCTCGAAAACACTTAAAGAC-3′ (SEQ ID NO: 11)) 363-bp PCR fragments were generated, digested within flanking SpeI and BamHI restriction sites, and cloned into the appropriate sites of the respective vectors. To generate pPBSMutMLEVleaderAkv-pac, a BstBI-BamHI-digested PCR fragment containing PBSMut and 465-bp MLEV-Psi was cloned into pPBSMut476PsiAkv-pac containing BstBI within the PBSMut sequence and BamHI immediately downstream from Psi.

In Vivo Analysis:

The general cell culture, transfection and transduction procedures are described Lovmand et al., Growth and purification of Murine Leukemia Virus. In “Cell Biology: A Laboratory Handbook”. Academic Press, Inc., 1994. Briefly, a mixture of 10 μg of vector plasmid, and 1 μg of pEGFP was transfected using the calcium phosphate precipitation method into BOSC23 packaging cells (Pear et al., Proc. Natl. Acad. Sci. U.S.A. 90: 8392-6, 1993) seeded at 7.1×10⁴ cells/cm² one day prior to transfection. The transfection efficiency was monitored by flow-cytometry. Virus containing media was serially diluted and in the presence of 6 μg/mL of polybrene (Sigma Chemical Co., St. Louise, U.S.A.) and transferred to NIH3T3 target cells. For selection of transduced target cells, medium containing G418 or puromycine was added to the NIH3T3 cells 48 hours post transfection. Resistant colonies were counted approx. 16 days post transduction and the resulting titer given as colony forming units per ml supernatant (CFU/ml)

Results:

TABLE 1
Transduction efficiency of vectors harboring alternative
palindrome in the kissing-loop:

| | Constructs: | titer (CFU/mL) |
|---|---|---|
| 1. | pPBSpro476PsiAkv-neo KL-altpal | 8.6 × 10⁴ |
| 2. | pMLEVleaderAkv-pac KL-altpal | 6.4 × 10⁴ |
| 3. | pPBSpro476PsiAkv-neo | 1.9 × 10⁵ |
| 4. | pMLEVleaderAkv-pac | 1.5 × 10⁵ |

The wildtype palindromic kissing-loop of the starting vectors pPBSpro476PsiAkv-neo and pMLEVleaderAkv-pac was substituted with an alternative palindrome (KL-altpal) and the transduction efficiency of the modified vectors was tested. The transduction efficiency of vectors harboring the alternative palindrome has decreased only approx. 2 fold (table 1).

TABLE 2
Increase in titer of PBS-mutated constructs upon
co-transfection of a rescue vector:

| | Construct A | Construct B | Rescue titer | Palindrome |
|---|---|---|---|---|
| | (PBS impaired vector) | (rescue vector) | increase (fold) | match |
| 1. | pPBSMut476PsiAkv-neo | pMLEVleaderAkv-pac | 44x | 6 of 6 |
| 2. | pPBSMutMLEVleaderAkv-pac | pPBSPro476Psi Akv-pac | 91x | 6 of 6 |
| 3. | pPBSMut476PsiAkv-neo KL-altpal | pMLEVleaderAkv-pac | 17x | 0 of 6 |
| 4. | pPBSMut476PsiAkv-neo KL-altpal | pMLEVleaderAkv-pac KL-altpal | 42x | 6 of 6 |

In the presented set-up the increase in rescue titer reflects the ability of the two vectors to recombine and heterodimerize. However, the experimental variation requires that experiments are conducted in parallel to allow comparison. The experiments listed in Table 2 have consequently been performed in parallel.

As seen in Table 2, pPBSMut476Psi Akv-neo may be rescued by co-transfection of the pMLEVleaderAkv-pac vector. Rescue titer increases 44 fold as compared to background level, ie. an individual titer determination of the constructs listed as construct A. When maximal complementarity exists between the palindrome of the rescue vector and the PBS mutated vector, the rescue titer is high (42-91 fold increase in comparison to background level, Table 2). However, when the wild type palindrome of the Akv-derived PBS mutated vector is substituted with an alternative palindrome (pPBSMut476Psi Akv-neo KL-altpal), the complementarity with the endogenous virus (pMLEVleaderAkv-pac) is destroyed (0 of 6 match) and the rescue titer is increased only 17 fold. Reconstituting the palindromic sequence in the rescue vector to match the alternative palindrome in the Akv-derived vector results in the restoration of the rescue titer to 42 fold that of the background level.

Conclusion:

In this set-up inserting the MLEV leader region into a vector construct mimics the expression of an endogenous murine leukemia virus (MLEV). Through recombination the vector construct is capable of rescuing an Akv-derived PBS-mutated vector with high efficiency. However, replacing the Akv kissing-loop sequence by an alternative palindrome results in a reduced efficiency of rescue. Thus, vectors comprising palindromic sequences have been designed that are able to transduce target cells with high efficiency and the designed vectors do not recombine as frequently as the vectors simulating endogenous retroviruses as they do with a vector with a matching palindrome.

### Example 3

### Introduction of Non-Palindromic Sequences into the Kissing Loop Region (DIS 2)

Introduction:

To study whether non-palindromic sequences can be used to direct the heterodimerization of retroviral vectors the following experiment was performed.

Description of Vector Constructs:

pPBSMutKL-nonpalAkv-neo and pMLEVleaderKL-nonpalAkv-pac: The non-palindromic loop motif (KL-nonpal) was introduced into the kissing-loop sequence of pPBSMut476Psi Akv-neo and pMLEVleaderAkv-pac (see example 2) by PCR-mediated mutagenesis using the following sense oligonucleotide matching Akv (the altered loop sequence is underlined): ON4 (5′-CTGATTCTGTACTAGTTAGGATACTAGATCGTATCTGGC-3′ (SEQ ID NO: 12)) introducing the non-palindromic sequence 5′-TAGGAT-3′ (SEQ ID NO: 13) together with an antisense oligonucleotide matching the most 3′ part (positions 617 to 638) of either Akv-Psi (ON8, 5′-CAGGTCGACGGATCCGTTTTTAGAAGCGGTCCAAAAC-3′ (SEQ ID NO: 10)) or MLEV-Psi (ON9, 5′-CAGGTCGACGGATCCGATCTCGAAAACACTTAAAGAC3′ (SEQ ID NO: 11)). PCR fragments of 363 bp were generated digested within flanking SpeI and BamHI restriction sites, and cloned into the appropriate sites of the respective resulting in pPBSMutKL-nonpalAkv-neo and pMLEVleaderKL-nonpalAkv-pac.

pMLEVleader KL-matchnonvalAkv-pac: was constructed using ON7 (5′-CTGATTCTGTACTAGTATCCTAACTAGATCTGTATCTGGC-3′ (SEQ ID NO: 14)) and ON9 for introduction of a non-palindromic loop sequences 5′-ATCCTA-3 KL-(matchnonpal) (SEQ ID NO: 15) complementary to the KL-nonpal sequence of the pPBSMutKL-nonpalAkv-neo.

In Vivo Analysis:

In vivo analysis was performed as described in example 2. The constructs were co-transfected into BOSC23 cells and titer experiments performed as described in example 2.

Results:

TABLE 1
Summary of rescue titer data:

| | | | Comple- | Rescue |
|---|---|---|---|---|
| | | | mentarity | titer |
| | | | kissing- | increase |
| | Construct A | Construct B | loop | (fold) |
| 1. | pPBSMutKL-nonpalAkv- | pMLEVleaderKL- | 0 of 6 | 68x |
| | neo | nonpalAkv-pac | | |
| 2. | pPBSMutKL-nonpalAkv- | pMLEVleader KL- | 6 of 6 | 147x |
| | neo | matchnonpalAkv- | | |
| | | pac | | |

For a thorough explanation of the set-up, see example 2 and FIG. 2c. In the first experiment (table 1) no complementarity exists between the two kissing-loops of the vectors. This gives rise to relatively low increase in rescue titer (68 fold) as compared to background level, i.e. an individual titer determination of the constructs listed as construct A. When restoring full complementarity between kissing-loops in experiment 2 (table 1) when co-transfecting the rescue vector (construct B) the rescue titer is increased (147 fold).

Conclusion:

The determinant for rescue of PBS mutated vectors, is complementarity between the sequences in the kissing loop region of the involved vectors. Thus, also the introduction of non-palindromic sequences into the kissing-loop region can be used for direction of heterodimerization of retroviral vectors.

### Example 4

### Compatibility of RNA I and -II with Akv-MLV-Derived Vectors

Introduction:

A major safety issue of employing retroviral vectors for gene transfer (gene therapy) is the risk of recombination between vector and endogenous virus, which may give rise to unwanted formation of replication competent viruses. The traditional murine leukemia-derived vectors (e.g. pPBSPRO 240 Akv neo) transduce target cells with high efficiency, however, they are prone to recombination with endogenous viruses. The rationale behind example 4 is to reduce the risk of recombination by the insertion of heterologous RNA elements that will impair heterodimerization between vector RNA and RNA from endogenous viruses. It is required that the heterologous RNA elements can be inserted in the vector without strongly affecting transduction efficiency. In the experiments described below, a region in Akv-based vectors has been defined for insertion of RNA elements of heterologous origin. At the defined position, the insertion of the tested RNA elements does not affect transduction efficiency. The sequences inserted in the presented experiments originate from the bacterial ColE1 plasmid, however, the results suggest that sequences with different origin can be inserted

Description of Vector Constructs:

pPBSPRO 240 Akv neo: is described in details in Lund et al., J. Virol. 67: 7125-7130, 1993. This vector is based on the Akv murine leukemia virus and contains in addition to the 5′ and 3′ LTR, primer binding site (PBS) and the first 240 nucleotides (nt) of the leader region and also the neo marker gene.

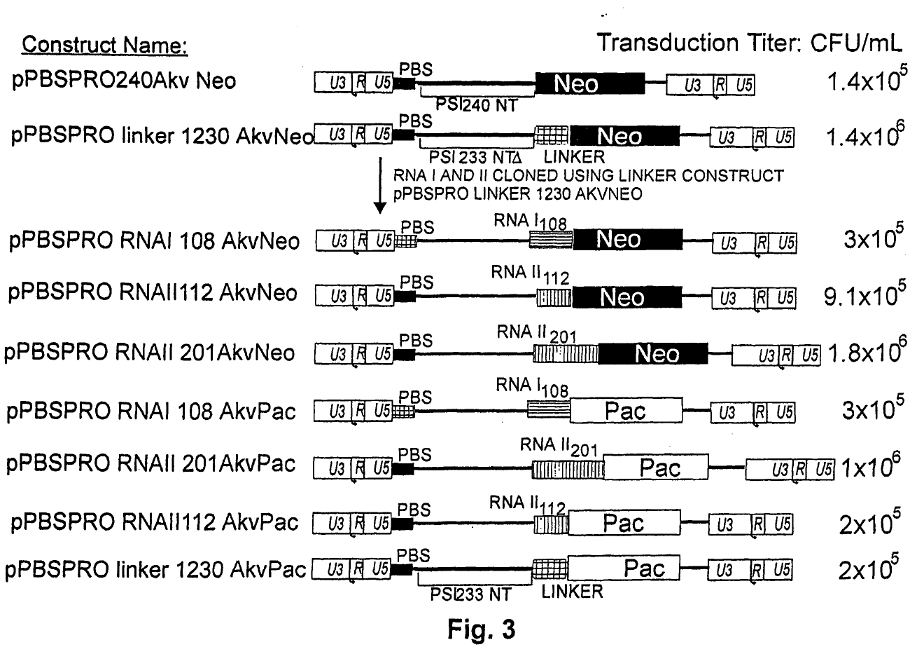

pPBSPRO 1230 linker Akv neo: Prior to insertion of the ColE1 elements RNA I or RNA II into the vectors, the pPBSpro linker 1230 Akv Neo vector was constructed, FIG. 3. A multiple cloning site (MCS) was designed and inserted directly downstream of the Akv-derived leader region of the pPBSPRO linker 1230 Akv Neo construct. Furthermore, 7 nucleotides were deleted from the 5′ end of the leader region, thereby reducing the leader region to 233 nucleotides. This was achieved by generating two PCR fragments: A: Upstream primer: 5′-CCGTCGGGAGGTAAGC 3′ (SEQ ID NO: 16) and downstream primer: 5′-CGTCGGATCCTACTACGCCTCGAGTGCCTCTGAGACGTCTCCCA 3′ (SEQ ID NO: 17) on a pPBSPRO240 Akv neo template. B: Upstream primer: 5′-ACTCGAGGCGTAGTAGGATCCGACGCAGCGGCCGCACCTACAGCCAAGCTTCACGCT3′ (SEQ ID NO: 18) and downstream primer: 5′-GGCGCCCCTGCGCTGACAGCCGGAACAC3′ (SEQ ID NO: 29) on a pPBSPRO244ψ Akv-Neo template. The two fragments were mixed and an overlap extension product generated with the upstream primer from fragment A and downstream primer from fragment B. The fragment was cloned into pPBSPRO240 Akv-Neo using standard techniques employing the SpeI-site in the leader region and the BclI-site in the neo gene, thereby generating pPBSPRO 1230-linker Akv neo.

pPBSPRO RNA I 108 Akv Neo, pPBSPRO RNA II 112 Akv Neo and pPBSPRO RNA II 201 Akv Neo: The ColE1 elements RNA I og RNA II have been inserted in the pPBSpro linker 1230 Akv Neo-vector, resulting in the vectors pPBSPRO RNA I 108 Akv Neo, pPBSPRO RNA II 112 Akv Neo and pPBSPRO RNA II 201 Akv Neo. The numbers accompanying the RNA elements (FIG. 3) 108, 112 and 201 refer to the size of the inserted elements. The RNA I 108 fragment consists of the full length RNA I transcript from the ColE1 plasmid (accession no. 301566). The RNAII-112 and -201 consists of the first 112 and 201 nucleotides of the RNA II transcript from the ColE1 plasmid, respectively.

To generate pPBSPRO RNAII 112 Akv neo and pPBSPRO RNAII 201 Akv neo, three PCR fragments were generated: C: Upstream primer: 5′-CGCGCGCTCGAGTGCAAACAAAAAACTGTAAGGAAAAAAGCGGCCGCACTGGTAACCGGATTAGCAGAGCGATGATGGCACAAACGGTGCTACAGAGTTCTGMGTAGTGGCCCGACTACGGCTACA3′ (SEQ ID NO: 20) on a pBR322 template (Accession 301749). Fragment D: Upstream primer: 5′-CGCGCGCTCGAGTGCAAACAAAAAAACACCGCTACCAACGGTGGTTTGTT-3′ (SEQ ID NO: 19) and downstream primer: 5′-TGTAAGGAAAAAGCGGCCGCACAGTATTTGGTTATCTGCGCT3′ (SEQ ID NO: 21) on a pBR322 template (accession 301749). Fragment E: Upstream primer: 5′-GATGTGCCGCTCGAGACAGTATTTGGTATCTGCGCT3′ (SEQ ID NO: 22) and downstream primer: 5′-TGTAAGGAAAAAAGCGGCCGCMCAAAAAAACCACCGCTACCAACGGTGGTTTGTTT-3′ (SEQ ID NO: 23) on a pPBR322 template. The three PCR fragments were gel purified and digested with XhoI (located in the upstream primer) and NotI (located in the downstream primer). These sites are also present in the MCS of the newly generated linker in pPBSPRO-1230-linker Akv neo and the fragments were cloned into this vector by standard cloning procedures. Consequently, the cloning of fragment C generated: pPBSPRO RNAII 201 Akv neo, fragment D generated: pPBSPRO RNAII 112 Akv neo and fragment E generated: pPBSPRO RNAI 108 Akv neo.

pPBSPRO RNA I 108 Akv pac, pPBSPRO RNA II 112 Akv pac, pPBSPRO RNA II 201 Akv pac and pPBSPRO 1230 linker Akv pac: are similar to the pPBSPRO Akv neo vectors described above except that the neomycin resistance gene (neo) has been substituted by the puromycin resistance gene (pac) The pac gene can be used as a dominant selectable marker to select for stably transformed mammalian cells in a manner analogous to the neo gene conferring resistance to G418. The pac gene was PCR-amplified from pPUR (Clontech Laboratories, Inc.) (Accession no. U07648). with primers containing NotI (5′-AGGATCCGACGCAGCGGCCGCACCATGACCGAGTACAAGCCCA (SEQ ID NO: 24)) and BsmI restriction sites (5′-TATCCAGATGAACAGCATTCTCAGGCACCGGGCTTGC) (SEQ ID NO: 25), respectively. The amplified 644-bp PCR product was digested with NotI and BsmI and inserted into NotI-BsmI-digested (restriction sites flanking neo) pPBS PRO linker 1230 Akv neo, pPBSPRO RNA I 108 Akv neo, pPBSPRO RNA II 112 Akv neo and pPBSPRO RNA II 201 Akv neo. Thus, generating the pPBSPRO 1230 linker Akv pac, pPBSPRO RNA I 108 Akv pac, pPBSPRO RNA II 112 Akv pac and pPBSPRO RNA II 201 Akv pac constructs.

In Vivo Set-Up:

The general cell culture conditions, transfection and transduction procedures are described in (Lovmand et al., Growth and purification of Murine Leukemia Virus. In “Cell Biology: A Laboratory Handbook”. Academic Press, Inc., 1994). Briefly, a mixture of 10 μg of vector plasmid, was transfected using the calcium phosphate precipitation method into BOSC23 cells (Pear et al., Proc. Natl. Acad. Sci. U.S.A. 90: 8392-6, 1993) seeded at 7.1×10⁴ cells/cm² one day prior to transfection. Virus containing media was serially diluted and in the presence of 6 μg/mL of polybrene (Sigma Chemical Co., St. Louise, U.S.A.) transferred to NIH3T3 target cells. For selection of transduced target cells, medium containing G418 or puromycin was added to the NIH3T3 cells 48 hours post transfection. Resistant colonies were counted approx. 16 days post transduction.

Results:

TABLE 1
Transduction titer (CFU/mL) of vector constructs
harbouring RNA I and -II elements:

| | Constructs: | titer (CFU/mL) |
|---|---|---|
| 1. | pPBSPRO 240 Akv neo | 1 × 10⁵ |
| 2. | pPBSPRO 1230 linker Akv neo | 1 × 10⁶ |
| 3. | pPBSPRO RNA I 108 Akv neo | 3 × 10⁵ |
| 4. | pPBSPRO RNA II 112 Akv neo | 9 × 10⁵ |
| 5. | pPBSPRO RNA II 201Akv neo | 2 × 10⁶ |
| 6. | pPBSPRO 1230 linker Akv pac | 2 × 10⁵ |
| 7. | pPBSPRO RNA I 108 Akv pac | 3 × 10⁵ |
| 8. | pPBSPRO RNA II 201 Akv pac | 2 × 10⁵ |
| 9. | pPBSPRO RNA II 201 Akv pac | 1 × 10⁶ |

The RNA I and -II elements were inserted into the pPBSPRO 240 Akv neo vector downstream of the Akv leader sequence, generating the constructs pPBS PRO linker 1230 Akv neo, pPBSPRO RNA I 108 Akv Neo, pPBSPRO RNA II 112 Akv Neo and pPBSPRO RNA II 201 Akv Neo. These vectors were tested for their transduction efficiency in a standard in vivo set-up Lovmand et al., Growth and purification of Murine Leukemia Virus. In “Cell Biology: A Laboratory Handbook”. Academic Press, Inc., 1994. All of the tested RNAI and -II containing neo-vectors gave rise to high transduction titers, comparable to or higher than the starting vector pPBSPRO 240 Akv neo. Analogous to the neo containing vectors, pac gene containing vectors were also generated: pPBSPRO 1230 linker Akv pac, pPBSPRO RNA I 108 Akv pac, pPBSPRO RNA II 112 Akv pac and pPBSPRO RNA II 201 Akv pac. These were shown to transduce target cells with high efficiency and the titers were not affected by the insertion of the RNAI and -II elements (table 1).

Conclusion:

Akv-derived vectors were constructed which harbored the ColE1 RNA dimerization elements, RNA I and -II, without affecting vector transduction efficiency as compared to the starting vectors.

### Example 5

### An Assay to Determine the Ability of RNA Elements to Facilitate Heterodimerization of Vector RNA

Introduction:

In future retroviral vector set-up, it could be of interest to generate virus particles containing a genome of two different RNAs. This would allow the transduction of larger genes by using both strands as a template for reverse transcription. To produce virus particles containing heterodimeric RNA, the vectors must harbor elements promoting heterodimerization with high efficiency. Furthermore, the generation of heterodimers can also be used in standard retroviral vector systems in which the viral dimerization elements can be substituted with non-viral elements. Consequently, the vector dimerization is solely facilitated through foreign sequences of non-viral origin. Insertion of foreign sequences promoting heterodimerization would override similarities between the vector heterodimerization elements and dimerization elements found in endogenous viruses. Hence, vectors may be designed that transduce target cells solely due to the heterodimerization characteristics of vector RNA, thereby strongly reducing or eliminating the risk of co-packaging of vector RNA with RNA transcripts from endogenous or exogenous viruses.

In order to exploit such dimerization elements the efficiency of heterodimerization needs to be determined. The assay described below in which transduction relies on virus particles containing heterodimeric RNA (heterozygous genome) may serve this purpose

The Strand-Transfer Assay—Selection for Heterodimers:

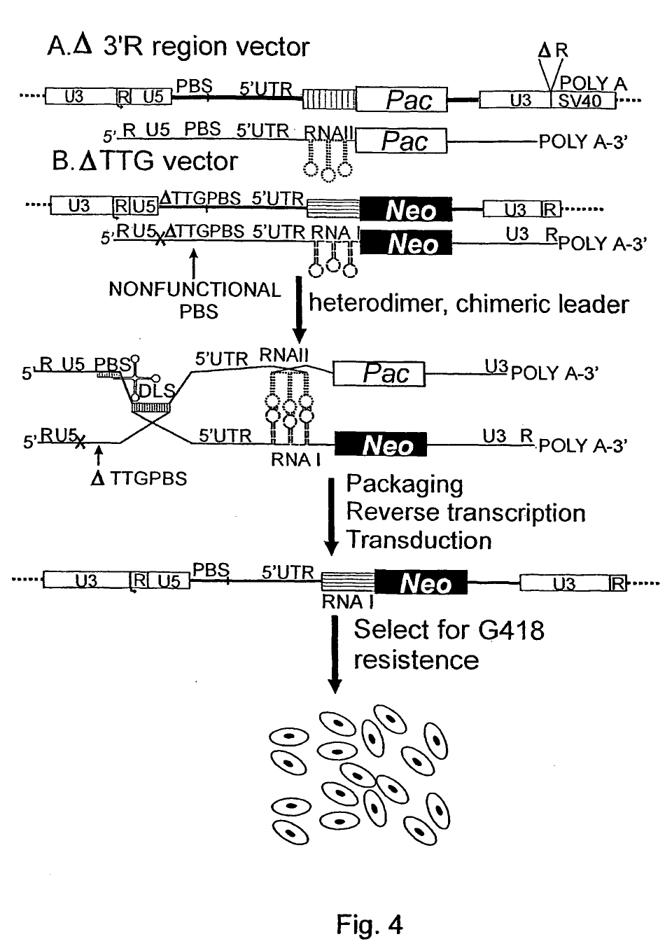

The set-up is outlined in FIG. 4. Heterodimerization elements are in the FIG. 25 exemplified by the RNA I and -II elements (see examples 4 and 6), however, any two sequences promoting heterodimerization may be inserted in the vectors. A functional PBS region is a requirement for a vector to proceed through the process of reverse transcription. Deletion of three 5′end nucleotides of the PBS (ΔTGG-vector) results in the reduction of the transduction titer by ≈10⁵ fold (see supplementing results, example 5) which is caused by impaired initiation of reverse transcription. For first strand transfer, complementarity between the R regions is required which has been demonstrated by experiments performed by (Dang and Hu, J. Virol. 75: 809-820, 2001) in which a homology of only three nucleotides between two R regions reduces the transduction titer 20 fold.

In the proposed assay, vectors are constructed harboring either the PBS deletion (ΔTGG-vectors) or the 3′R region deletion (Δ3′R-vectors) which presumably results in impaired transduction ability. However, co-transfecting packaging cells with two vectors, one vector with each type of mutation, the vector RNA can co-package in the virion. The virion thereby contains one intact PBS for initiation of reverse transcription and one intact R region for first strand transfer. When 1'st strand synthesis is initiated on the strand carrying the intact PBS, the second strand transfer must be intermolecular as the other strand is carrying the intact 3′ R region. The 1'st strand synthesis proceeds on this strand and finally reverse transcribe the ΔTGG-PBS. Although the ΔTGG-PBS cannot facilitate initiation of 1'st strand synthesis, it can facilitate 2'nd. strand transfer as it is complementary to 15 of the 18 nucleotides transcribed from the PBS. Consequently, there are no demands of an interstrand template shift during 1'st strand synthesis (recombination) as is the case for the assay described in the assay described in example 2. Thus, virus particles containing the heterodimeric (heterozygous) genome are able to transduce target cells with higher efficiency than viruses containing a homozygous genome. Consequently, the transduction titer of ΔTGG-vectors comprising the selection gene may be a quantitative measure of the effectively the vectors are guided together and co-packaged as heterodimers.

The assay system can be employed in the testing heterodimerization of any RNA element directing heterodimerization.

Supplementing Results:

The ΔTGG-vectors have been tested in an in vivo set-up. The set up in BOSC23 cells is described in example 2 and the construction of the vectors is described in example 6.

TABLE 1
Transduction efficiency of pPBSΔTGG Akv neo:

| | Construct name | transduction titer (CFU/mL) |
|---|---|---|
| 1. | pPBSPRO Akv neo | 5 × 10⁵ |
| 2. | pPBSΔTGG Akv neo | 10¹ |

### Example 6

### RNA I and RNA II Containing Constructs—Directing Specificity of Packaging

Introduction:

In example 4, it is shown that heterologous RNA elements can be inserted in a given position of an Akv-derived vector without affecting transduction efficiency. As described in the introduction to example 5 it is of interest to construct vectors that transduce through heterodimerization. When designing elements that promote heterodimerization, their ability to direct formation heterodimers needs to be evaluated in a biological set-up. In example 5, an assay was described in which the transduction efficiency is measured through heterodimerization. A more direct assay is presented in this example in which the RNA contents of the virus particles is determined.

All retroviruses contain an RNA element that is necessary for packaging (PSI element) of the retroviral genome into the virus particle. When removing the packaging signal from a vector only small amount of RNA is packed. However, if RNA lacking the packaging signal is linked to a second RNA in a dimer, the RNA lacking the packaging signal can rely on the packaging signal contained in the other RNA strand. This way packaging of an RNA devoid of the packaging signal with an RNA containing a packaging signal is a measure of their ability to form heterodimers. Such an assay is used to evaluate the ability of RNA I and -II to direct specific heterodimerization. RNA I is inserted in a vector lacking a packaging signal and RNA II elements in a vector containing a packaging signal.

Description of Vector Constructs:

pPBSproΔTGG Akv neo: For ease of subsequent cloning of mutated PBS sequences restriction sites for Srf1 and Nru1 were created in pPBSpro at position 952 and 1018, respectively, at which half sites for Srf1 and Nru1 exist. A DNA fragment containing restriction sites for Srf1 and Nru1 was obtained by two-step PCR and overlap extension. 150 ng pPBS pro was used as template for a PCR reaction (PCR 1) in which 25 pmol primer 148760 (5′-AGCTTACCTCCCGACGGTGGGTCGCGATGTGTGTGTGGCCCGGGCAGTCAATCACTCTGAGGA (SEQ ID NO: 26)) and 25 pmol primer A6600 (5′-CAGCTTGTCTGTMGCGGATGC (SEQ ID NO: 27)) were used under standard PCR reaction conditions (15 cycles: 94° C. for 1 min; 60° C. for 1 min; 73° C. for 2 min. followed by 73° C. for 5 min). For PCR 2, the PCR conditions were identical except the primers were replaced by 25 pmol primer 147654 (5′-GACCCACCGTCGGGAGGTAAGCT (SEQ ID NO: 28)) and 25 pmol primer 137599 (5′-GGCGCCCCRGCGCTGACAGCCGGAACAC (SEQ ID NO: 29)). PCR 1 and PCR 2 resulted in 930 bp and 720 bp products, respectively. The two PCR fragments were combined in an overlap extension reaction using 200 ng each PCR product 1 and 2 under standard PCR conditions but also including 1.25 U of Pfu 1. The following cycling parameters were used: 73° C. for 5 min; and five cycles consisting of: 94° C. for 1 min; 60° C. for 2 min; 73° C. for 2 min., followed by 73° C. for 5 min. 10 pmol each end primer A6600 and 137599 were added to the reaction and cycling repeated. The resulting 1650 bp product was purified by gel electrophoresis and extracted. The product was digested with EcoRI and BamHI and inserted into the pPBSpro cut with EcoRI and BamHI by standard ligation, resulting in the construct pPBSpro Srf1/Nru1. A DNA fragment comprising the PBS in which the TGG sequence had been deleted was obtained by mixing 100 pmol each oligonucleotide 150500 (5′-P-CTGCCCAGCCTGGGGGTCTTTCATTGGGCTCGTCCGGGAT (SEQ ID NO: 30)) and 150501 (5′-P-CGGTGGGTCGGTGGTCCCTGGGCGGGGGTCTCCAAATCCCGGACGAGCCCAATGAAAGACCCCCAGGCT (SEQ ID NO: 31)). The oligonucleotides were heated to 95° C. for 5 min and reannealed by slow cooling to room temperature. pPBSpro Srf1/Nru1 was digested with Srf1 and Nru1. The 3′ terminal sequence of the digested vector were removed by the 3′ to 5′ exonuclease activity of T4 DNA polymerase in the presence of final concentration of 0.5 mM dATP (as described in Sampath et al., Gene 190: 5-10, 1997; Aslanidis and Jong, Nucl. Acids Res. 18: 6089-74, 1990). The DNA fragment was inserted into the vector by standard ligation, resulting in pPBSproΔTGG Akv neo.

pPBSΔTGG linker 1230 Akv neo, pPBSΔTGG RNA I 108 Akv neo, are based on the vector constructs pPBSPRO linker 1230 Akv neo, pPBSΔTGG RNA I 108 Akv neo and pPBSΔTGG Akv neo, see example 4. A 732 bp DNA fragment harbouring the modified PBSΔTGG was isolated after EcoRI and SpeI digestion of pPBSΔTGG Akv neo and cloned into SpeI and EcoRI digested pPBSPRO linker 1230 Akv neo and pPBSΔTGG RNA I 108 Akv neo. This generated the pPBSΔTGG linker 1230 Akv neo and pPBSΔTGG RNA I 108 Akv-neo vectors.

pPBSΔTGGΔ Psi linker 1230 Akv neo. pPBSΔTGGΔ Psi RNA I 108 Akv neo: The pPBSΔTGG linker 1230 Akv neo, pPBSΔTGG RNA I 108 Akv neo, was cut with SpeI and XhoI. The DNA was incubated with dNTPs and Klenow enzyme to fill out the overhang, and subsequently religated with T4 DNA ligase. This gave rise to a 89 nucleotides deletion of the core packaging signal. Thus, according to literature the sequences needed for efficient packaging were been removed (Mougel and Barkils, J. Virol. 71: 8061-5, 1997).

pPBSPRO RNA II 112 Akv pac, pPBSPRO RNA II 201 Akv pac and pPBSPRO 1230 linker Akv pac: The construction of these vectors are described in example 4.

In Vivo Analysis of Vector Constructs:

Plat-E packaging cells (Morita et al., Gene Ther. 7: 1063-6, 2000) were double transfected with 10 μg of each construct A and B as shown in Table 1. To monitor the transfection efficiency 1 μg EGFP was co-transfected and the level of EGFP expression was determined by flow cytometry. From the transfected cells, virus supernatant was collected and virus isolated as described in (Lovmand et al., Growth and purification of Murine Leukemia Virus. In “Cell Biology: A Laboratory Handbook”. Academic Press, Inc., 1994.). The isolated viruses were split in two pools. Pool A for quantification of virus amount by reverse transcriptase assay (Lovmand et al., Growth and purification of Murine Leukemia Virus. In “Cell Biology: A Laboratory Handbook”. Academic Press, Inc., 1994). Pool B: Viruses for a whole-virion dot blot assay (Nelson et al., Hum. Gene Ther. 9: 2401-5, 1998).

Analysis of Virion RNA Contents by Dot Blot Analysis:

The RNA content of the virus particles is linked to a filter (Zeta-Probe, Blorad, Hercules, U.S.A.). The dot blot filter was subsequently probed with a Neo-probe in order to quantify RNA derived from the packaging signal deficient vectors. By standardizing to the amount of viruses (measured by RT-assay (Lovmand et al., Growth and purification of Murine Leukemia Virus. In “Cell Biology: A Laboratory Handbook”. Academic Press, Inc., 1994) It was determined to which degree RNA I and RNA II could direct packaging. A summary of the data is shown in table 1.

Quantification of the hybridization signal of the dot blot filter was performed as the intensity units multiplied by area (CNTmm2) and thus represent the raw data for packaging. The RT activity was measured as the incorporation of [H³] dTTP and represents the amount of viruses analyzed in each experiment. Relative packaging is shown in the last column to the right. The relative packaging is calculated as the raw RNA contents relative to the amount of virus particles.

TABLE 1

| | | | Transfection | Raw packaging | Virus load | |
|---|---|---|---|---|---|---|
| | | | efficiency | data (Neoprobe) | Standardized | Relative |
| Exp | Construct A (10 μg) | Construct B (10 μg) | 1 μg EGFP | CNT mm2 | (RT-assay) | packaging |
| 1 | Linker 1230 Δtgg | Linker 1230 pac | 49 | 13286 | 1 | 1 |
| | ΔΨ neo | | | | | |
| 2 | Linker 1230 Δtgg | RNA II 112 pac | 51 | 4309 | 0.7 | 0.3 |
| | ΔΨ neo | | | | | |
| 3 | RNA I 108 Δtgg ΔΨ | RNA II 112 pac | 52 | 25897 | 1.3 | 2.7 |
| | neo | | | | | |
| 4 | RNA I 108 Δtgg ΔΨ | RNA II 201 pac | 53 | 24806 | 1.8 | 3.3 |
| | neo | | | | | |

Results:

Experiment 1 is a control experiment presenting data for vector constructs in which the RNA I and II elements have not yet been inserted. Thus, the packaging measured (1.0) is a result of background packaging in the test system. Also experiment 2 is a control in which only a RNA II element is included in one of the vectors. This insertion is, however, not sufficient for directing packaging of the pPBSΔTGGΔPsi linker 1230 Akv neo (0.3). In experiment 3 and 4 vectors contain complementary RNA I and -II sequences which give rise to increased relative packaging (2.7 and 3.3, respectively)

Conclusion:

Complementary RNA I and -II elements inserted in the two vectors can direct specificity of packaging and dimerization.

### Example 7

### Full-Length Viruses Harbouring Alternative Palindromes in a Potential Kissing-Loop Sequence Positioned Upstream of the Core Packaging Signal are Able to Replicate

Introduction:

Moloney murine leukemia virus (Mo-MLV) comprises two RNA stem loops known as the dimer initiation site (DIS) DIS 1 and DIS 2 at position 204 to 228 and 283-298, respectively. Studies in which the DIS 1 and DIS 2 were systematically mutated revealed that the two DIS elements together form a bipartite organization assisting in the initiation of dimer assembly in the virus (Ly and Parslow, J. Virol. 76: 3135-3144, 2002). In Akv murine leukemia virus a 10 nt palindromic region (pal-1) homologous to the DIS 1 identified in Mo-MLV is present at position 209-218 upstream of the core packaging signal comprising pal-2 (the DIS 2 homolog) and two GACG stem loops. Pal-1 is potentially involved in dimerization though a kissing-loop mechanism as suggested for DIS 1 and 2. In the present experiment the effect of alternative palindromes inserted in the potential kissing loop sequence pal-i (DIS 1) has been investigated.

Description of Virus Constructs:

PCR mutagenesis was employed to modify the DIS 1-like palindromic region (pal-1) in AkvB. AkvB is a B-tropic derivative of Akv-MLV modified at CA position 110, and it is isogenic to AkvBU3-EGFP (Aagaard et al, J. Gen. Virol., 83: 439-442. 2002) besides lacking the IRES-EGFP cassette positioned at the Cell/II site in the U3 region of AkvBU3-EGFP The palindrome 5′-GCTGGCCAGC-3′ (SEQ ID NO: 32) at position 209-218 in Akv/AkvB was modified at the at the six central positions to the alternative palindrome 5′ GCACCGGTGC 3′ (SEQ ID NO: 33) (changes are underlined) as follows:

A 1382 bp PCR fragment amplified from the AkvB plasmid using upstream primer A: 5′-GGGTCTGACGCTCAGTGGAAC-3′ (SEQ ID NO: 34) (located in the backbone at position minus 1140 relative to the Akv genome and upsteam the AsnI site at position minus 747) and the downstream primer B: 5′-GACAGAGACGGAGACAAAACGATCGCACCGGTGCTTACCTCCCGACGGTG-3′ (SEQ ID NO: 35) (spanning position 242-193 in Akv and harbouring six nucleotide mismatches in the pal-1 region as underlined) was used in a PCR overlap extension reaction with a 184 bp PCR fragment amplified from AkvB using upstream primer C: 5′-GATCGTTTTGTCTCCGTCTCTGTC-3′ (SEQ ID NO: 36) (spanning position 219-242 in Akv and with a 24 nt overlap to primer B) and downstream primer D: 5′-GCCCCCGATGCCTCTGAGACGTCTC-3′ (SEQ ID NO: 37) (spanning position 403-379 in Akv and located downstream the SpeI site at position 302) in a PCR reaction using primers A and D. The resulting 1542 bp PCR fragment and the AkvB plasmid were digested with AsnI and SpeI, and the resulting fragment was subsequently cloned into the linearized AkvB plasmid to generate AkvB-altpal using standard cloning procedures.

In Vivo Analysis:

10 μg of the plasmids encoding the replication-competent viruses was transfected by the calcium phosphate precipitation method into BOSC23 packaging cells (Pear et al., Proc. Natl. Acad. Sci. U.S.A. 90: 8392-6, 1993). The resulting virus supernatant was used to infect BALB/c fibroblast cells (ATCC CCL 163) seeded at 1000 cells per cm² one day prior to infection in the presence of 6 μg polybrene per ml (Sigma Chemical Co., St. Louise, U.S.A.). Two days post infection (dpi) the number of infected cells was quantified by Flow cytometry using a specific rat anti-envelope monoclonal antibody (83A25, Sitbon, et al. Virol 141: 110-118.1985) and a phycoerythrin (PE) conjugated goat anti-rat immunoglubulin (Harlan Sera-Lab LTD, Loughborough, England) for detection of envelope expressing cells. Non-infected BALB/c cells served as negative control for background PE signal. A parallel culture of BALB/c cells was allowed to divide until confluence (5 dpi) and the number of infected cells was re-quantified. The relative increase in the number (as measured in percentage) of infected cells from 2 to 5 dpi. is used as a measurement of replication kinetics.

Results:

The AkvB-altpal virus comprising the alternative palindromic sequence at position 209-218 was tested along with the parental (wildtype) virus AkvB for the ability to replicate in murine BALB/c fibroblasts. As shown in table 1 no significant difference in the replication capabilities between AkvB and AkvB-altpal was detected. Note that the relative increase varies between the two experiments. This is most likely due to differences in the total number of cell divisions during individual experiments and thereby the number of replication cycles the virus can undergo before blocked by lack of cell division.

TABLE 1
Replication kinetics of AkvB virus comprising an
alternative pal-1 sequence: Percentage of infected
cells at 2 or 5 days post infection (dpi) is shown
along with the relative increase.

| | Exp I | | | Exp II | | |
|---|---|---|---|---|---|---|
| | | | Relative | | | Relative |
| Virus construct | 2 dpi | 5 dpi | increase | 2 dpi | 5 dpi | increase |
| AkvB | 7.4% | 35% | ˜5 fold | 5.0% | 72% | ˜14 fold |
| AkvB-altpal | 4.3% | 23% | ˜5 fold | 3.1% | 50% | ˜16 fold |

Conclusion:

The pal-1 corresponding to position 209-218 of Akv murine leukemia virus may be replaced with an alternative palindromic sequence without affecting the replication capabilities of the virus. From the data presented in (Ly and Parslow, 3. Virol. 76: 3135-3144, 2002 and Oroudjev et al., J. Mol. Biol. 291: 603-13, 1999) it is clear that the pal-1 sequence promotes dimer formation. Therefore, replacing the wildtype palindrome of Akv with an alternative palindrome, without affecting the replication, strongly suggests that the alternative pal-1 promote homodimerization.

### Example 8

### Construction of Vectors Harboring Multiple Alterations in Dimerization Promoting Elements

Introduction:

In example 2, it was demonstrated that recombination with a specific endogenous retroviral sequence could be reduced in Akv-derived vectors with alternative palindromic sequence inserted into the kissing-loop region (DIS 2) without compromising transduction efficiency. It has also been demonstrated that both vectors and full-length viruses could be modified in a similar palindromic sequence (pal-1/DIS 1) and that when the modified sequence conserved the palindrome, both vectors (Mo-MLV-derived, Ly and Parslow, J. Virol. 76: 3135-3144, 2002) and full-length viruses (Akv-MLV, example 7) replicated close to wildtype level. The modified region (DIS 1) has furthermore been shown by in vitro experiments to be involved in dimerization (Ly and Parslow, J. Virol. 76: 3135-3144, 2002 and Oroudjev et al., J. Mol. Biol. 291: 603-13, 1999).

When a single dimerization signal is modified the vector derived RNA may heterodimerize with RNA transcripts of endogenous viruses through the interaction of remaining native dimerization signals contained within both vector and endogenous viral RNA. Therefore, it may be possible to design vectors similar to the vectors described in example 2 that are not only modified in the DIS 2 region but also remaining dimerization regions such as the DIS 1 region. From the data presented in examples 2 and 7 such multiple modified vectors may transduce target cells efficiently. Importantly, the specificity towards homodimerization would be increased by modifying two or more dimerization signals as the similarity to native dimerization elements within endogenous or exogenous viruses decreases.

In the rescue system described in example 9 a novel DIS 1 and DIS 2 double-modified vector would probably not be rescued to the same extent as a non-modified vector (derived from wildtype vector sequence) or single-modified vector (as the DIS 2 modified vector from example 2). Similarly, in a two vector system as described in example 2, a double-modified vector (DIS 1+DIS 2) with an impaired PBS would result in further reduction in rescue titers as compared to the non-modified vectors or the single modified DIS 2 vector.

For retroviral vectors derived from other groups of retroviruses, such as lentiviruses, it may similarly be of interest to design vectors comprising alterations in multiple motifs involved in dimerization.

### Example 9

### Recombinatorial Rescue Assay for Mobilization of PBS-Mutated Vector Constructs Achieved by a Full-Length Virus through Heterodimerization

Introduction:

Reverse transcription of a retroviral RNA is initiated from a cellular derived tRNA molecule, which specifically binds to the viral primer-binding site (PBS). Reverse transcription of the PBS-modified vectors is impaired and the transduction of target cells drastically reduced (Lund et al., J. Virol 71: 1191-1195, 1997; Hansen et al., J. Virol. 75: 4922-4928, 2001 and U.S. Pat. Nos. 5,886,166, 5,866,411, 6,037,172 and 6,107,478 all of which are incorporated herein by reference). However, co-expressing an artificial tRNA matching the mutated primer-binding site in the packaging cells restores the transduction of target cells to nearly wild type level (Lund et al, J. Virol 71: 1191-1195, 1997; Hansen et al., J. Virol. 75: 4922-4928, 2001). The described tRNA complementation system controlling the initiation of reverse transcription can be employed in the current in current retroviral vector systems to promote a safe use of the retroviral vectors, as the risk of forming replication competent vectors through recombination is reduced. However, in target cells the transduced vector may be repaired if the target cells independently of the gene transfer are infected with a replication-competent virus.

In brief, packaging cells are transfected with neo-gene containing vectors and the virus-containing supernatant used to transduce target cells. For transduction of PBS-impaired vectors a complementing synthetic tRNA is co-transfected into the packaging cells. Transduced cells are selected for and re-seeded. Re-seeded vector bearing cells are infected with a full-length replication competent Akv murine leukemia virus. The supernatant of these cells is transferred to target cells that are subjected to selection. Emerging cell colonies are in case of PBS-mutated vectors the result of co-packaging of neo vector and full-length virus RNA as Akv-MLV provides a functional PBS but also the result of recombination between the PBS-modified vectors and Akv in order to complete reverse transcription. Directing the heterodimerization and thus co-packaging by employing alternative dimerization elements in the used vectors may represent a novel safety feature. This recombinatorial rescue assay allows quantitative assessment of the impact of such alternative dimerization elements on the risk of recombinatorial rescue by an endogenous or exogenous retrovirus. Moreover, such PBS-modified vectors with Psi-modifications that cause reduced recombinatorial rescue may have direct applications due to their improved safety profile.

Description of Vectors:

PPBSx2MLEVPsi Akv-neo: Is constructed form the pPBSGlnMLEVPsi Akv-neo described in example 2. Two PCR products were generated. PCR product A: using primer ON1 (5′-GCAGACCCCTGCCCAGGGACCACC-3′ (SEQ ID NO: 38)) and ON2 (5′GGCGCCCCTGCGCTGACAGCCGGAACAC-3′ (SEQ ID NO: 29)) on pPBSGlnMLEVPsi Akv-neo template. PCR product B: using primer ON3 (5′-GGGAATTCTACCTTACGTTTCCCCGACCAGAGCTGAATGTTCACAG-3′ (SEQ ID NO: 39)) and ON4 (5′GGTGGTCCCTGGGCAGGGGTCTCC-3′ (SEQ ID NO: 40)) on a pPBSx2 Akv-neo template, described in (Lund et al, 3. Virol 71: 1191-1195, 1997). PCR product A and B was gel purified. A third PCR product C, was generated by in a overlap PCR using ON5 (5′GGGAATTCTACCTTACGTTT-3′ (SEQ ID NO: 7)) and ON6 (5′CTTCCTTTAGCAGCCCTTGCGC-3′ (SEQ ID NO: 41)) on a template consisting of a mixture of PCR products A and B. PCR product C was purified and cut with restriction enzymes EcoRI and BamHI and cloned into a EcoRI and BamHI digested pPBSProAkvΔPsi plasmid (described in Mikkelsen, 3. Gen. Virol. 80:2957-67.1999), by standard cloning procedures. This generated the pPBSx2MLEVPsi Akv-neo plasmid harbouring a modified PBS and a MLEV packaging signal.

pPBSGlnMLEVPsi Akv-neo: Construction is described in example 2.

In Vivo Analysis:

10 μg of each vector construct was transfected into BOSC23 packaging cells (Pear et al., Proc. Natl. Acad. Sci. U.S.A. 90: 8392-6, 1993) using the calcium phosphate precipitation method. In the case of pPBSGlnMLEVPsi Akv-neo 5 μg of pUC19 was included into the transfection mixture, whereas for the pPBSx2MLEV Akv-neo vector transfections were supplemented with either 5 μg of pUC19 (no complementation) or 5 μg of ptRNAx2 encoding an artificial tRNA (complementation as described in Lund et al, 3. Virol 71: 1191-1195, 1997). Supernatant was used to transduce NIH 3T3 fibroblasts and G418 resistant colonies were subsequently counted and selected as described in Lovmand et al. (1994). Resistant colonies, harboring the vector pPBSGlnMLEVPsi Akv-neo were pooled whereas for pPBSx2MLEV Akv-neo-transduced NIH cells six individual colonies were picked and expanded. NIH cells from the pool and the six clones were re-seeded at 5000 cells per cm² and super-infected with full-length replication-competent Akv virus (Etzerodt et al., J. Virol 134: 196-207, 1984) in the presence of 6 μg polybrene per ml (Sigma Chemical Co. St. Louise, U.S.A.). Seven days post infection (dpi) supernatant from the confluent cells was collected and neo titers were determined by transduction of new NIH cells. The neo-gene bearing NIH colonies arising after mobilization of pPBSx2MLEV Akv-neo was individually picked and expanded. Genomic DNA was prepared (DNAzol, Molecular Research Center Inc, Cincinnati, U.S.A.) for PCR and sequencing using primers flanking the PBS-leader region (upstream primer U3 5′-GCGGCCGCGATTCCCAGATGACCGGGGATC-3′ (SEQ ID NO: 42) and downstream primer neo 5′-GGCGCCCCTGCGCTGACAGCCGGAACAC-3′ (SEQ ID NO: 29)).

Results:

NIH cells were transduced with the pPBSGlnMLEVPsi Akv-neo vector produced in BOSC23 cells at high efficiency (titer 5×10⁵ CFU per ml) whereas pPBSx2MLEVPsi Akv-neo gave a low titer of ˜2 CFU per ml. However the low transduction of the PBS mutated vector could almost completely be restored by co-transfection with an artificial tRNA (ptRNAx2). Titers were increased 6500 fold to 1.3×10⁴ CFU per ml.

TABLE 1
Transduction titers of vector mobilized from NIH fibroblasts
upon infection with full-length Akv murine leukemia virus:

| | Virus | Titers |
|---|---|---|
| Vector present in fibroblast | challenge | (CFU per ml per 5 × 10⁶) |
| PPBSGlnMLEVPsi Akv-neo | Akv-MLV | 9 × 10³ |
| PPBS×2MLEVPsi Akv-neo | Akv-MLV | 1 × 10¹ |

Note that titers from PPBS×2MLEVPsi Akv-neo cells represent an average from six clones whereas the PPBSGlnMLEVPsi Akv-neo cells were polyclonal.

According to table 1 the intact vector pPBSGlnMLEV Akv-neo is efficiently rescued to untransduced cells via packaging into Akv virus particles. The PBS mutated vector pPBSx2MLEV Akv-neo can also be rescued albeit at a reduced efficiency. Rescue of PBS-impaired vectors results from co-packaging and recombination with Akv-MLV.

Sequence analysis of mobilized PBS mutated vectors has revealed that all transduced proviruses were a results of recombination events between the pPBSx2MLEV Akv-neo vector and Akv virus. Analysis of the recombination junction site show that 46% (16/35) coincide with the pal-2 (DIS 2) region. This is in agreement with previously reported hotspot recombination between Akv-derived vectors and endogenous retroviral RNA (Mikkelsen et al, J. Virol. 70:1439-47, 1996) and furthermore argues that mobilization involves heterodimerization.

Conclusion:

The recombinatorial rescue assay can be used to measure the efficiency of mobilization of PBS-impaired vectors and thus evaluate the ability of leader modified vectors to heterodimerize and co-package and recombine with replication-competent viruses.

### Example 10

### Affinity Purification of Retroviral RNA Dimers from Virus Particles for Analysis of Homodimer and Heterodimer Formation

Introduction:

RNA vector transcripts expressed in a packaging cell can form heterodimers with endogenous viruses expressed in the same cell. This is a major safety issue, as formation of such heterodimers will result in co-packaging of vector construct with endogenous virus. During reverse transcription of the heterodimeric RNA, recombination can occur and result in the generation of novel replication competent viruses. When developing vectors that reduce the risk of recombination with endogenous viruses, it is of interest to employ an assay that in a biological system can evaluate the abilities of modified vectors to heterodimerize with RNA transcripts from endogenous viruses. In addition it can also be applied for testing the ability of non-viral sequences (e.g. ColE1) to form homodimers and heterodimers.

The Oligo-Tagging Assay:

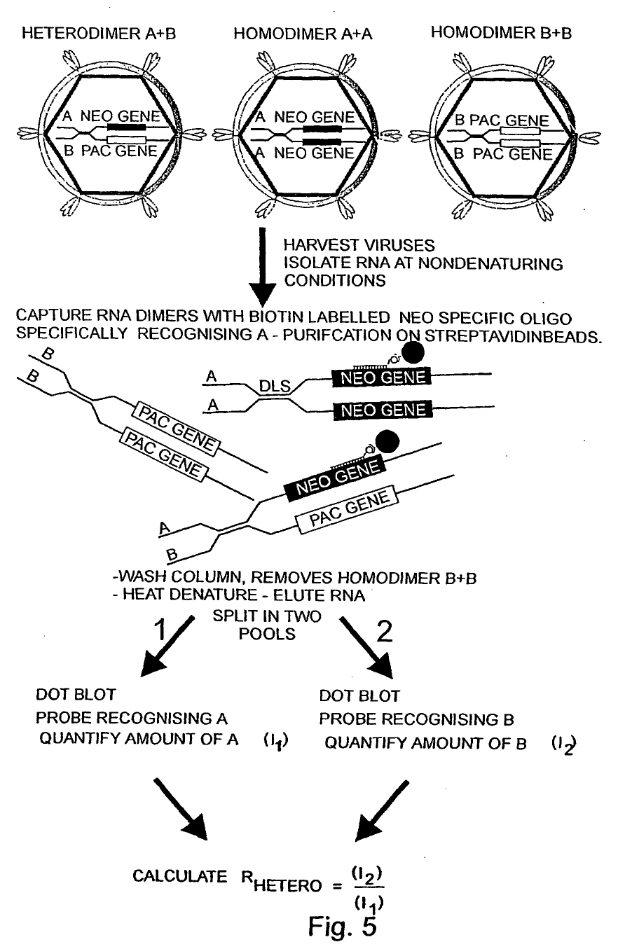

A packaging cell line is transfected with two construct of interest. To distinguish the two constructs they must harbor a stretch of unique sequence. The two transfected constructs would typically be of vector modified in dimerization signals and/or sequence of endogenous virus origin. However any two sequences of interest can be examined for their abilities to form heterodimers in virions. For simplicity the following example is described for an experiment using an Akv-derived retroviral vector (A) harboring a neo selection gene and a MLEV-derived vector (B) harboring a pac selection gene (FIG. 5). The virus containing medium from the A+B transfected packaging cells are collected and viral RNA isolated at non-denaturing conditions. The isolated RNA dimers, constitutes a population of following dimer combinations: A+A, B+B and A+B. The dimeric RNA is added an oligo specifically recognizing a sequence in A. This oligo is covalently attached to a linker (e.g. blotin). Dimers to which the oligo has hybridized can then be fixed to a column (e.g. avidin). The column material is washed several times in an appropriate buffer and finally RNA is eluted by heat denaturation. The eluted RNA is split into to pools. Both pools are linked to probing filter by a dot-blot method or equivalent. The filter from dot blot 1 is then probed with a probe recognizing A. The filter from dot blot 2 is probed with a probe recognizing B. With use of a phosphorimager the dot blot 1 can give quantitative measure of the amount of A+A and A+B dimers (I₁) in the pool, and for dot blot 2 a quantitative amount of A+B dimers, I₂. The ratio, Rₕₑₜₑᵣₒ=I₂/I₁ will be a measure of heterodimerization efficiency. A low Rₕₑₜₑᵣₒ is equivalent to the formation of small amounts of heterodimers and vice versa. As a control RNA must be isolated from viruses produced from packaging cells single transfected with A and B. This results in RNA homodimers consisting of A+A and B+B and no heterodimers, A+B. The homodimers A+A, B+B are then mixed and submitted for the same analysis as described above. The Rₕₑₜₑᵣₒ for the control must be significantly larger than any Rₕₑₜₑᵣₒ measured for double transfected cells, thereby confirming that no heterodimers are formed during the oligo purification.

Conclusion:

This assay can be used to compare the ability of different sequences to direct homodimerization and/or heterodimerization.

### Example 11

### Immobilization of Retroviral Vectors Facilitated by Heterodimerization through Pairs of Matching Non-Palindromic Sequences

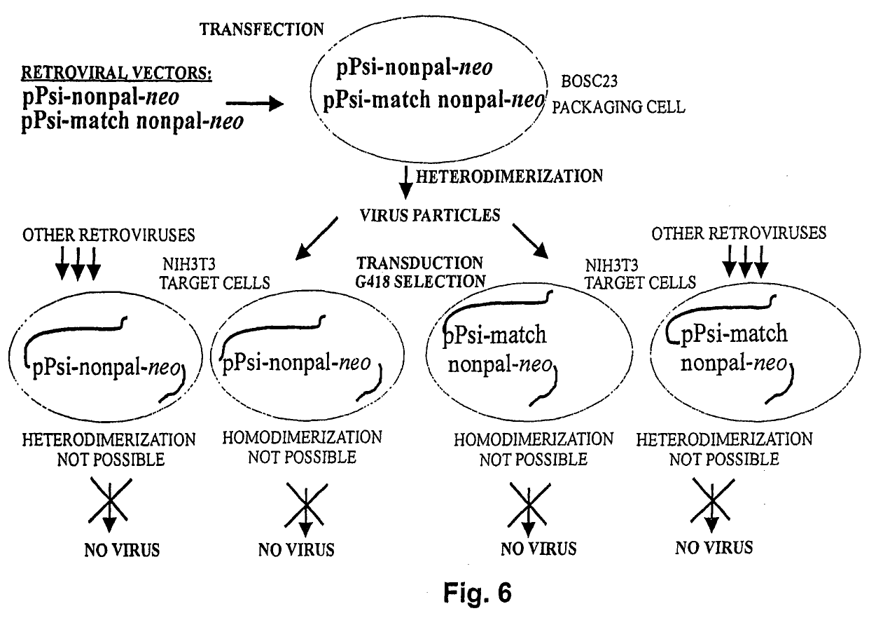

An additional safety feature of retroviral vectors can be offered by heterodimerization achieved by insertion of dimerization sequences into retroviral vectors as described in the following and illustrated in FIG. 6.

Two retroviral vectors are co-transfected into a packaging cell line. One vector is designed to contain a non-palindromic dimerization sequence and the design of the second vector is characterized by the insertion of a non-palindromic sequence, matching the non-palindromic element of the first vector. Virus particles that are released from the packaging cells will predominantly comprise RNA heterodimers of the two vector transcripts, as heterodimers will be formed with higher frequency than homodimers of each of the vector transcripts, due to the non-palindromic nature of the dimerization sequence. The non-palindromic and the matching non-palindromic sequences may be exemplified by KL-nonpal and KL-matchnonpal, respectively, as described in example 3). The resulting virus particles are used to transduce target cells. Only one provirus resulting from the process of reverse transcription of the RNA dimer will be integrated into the host cell genome Hence, the number of proviruses in the target cell will follow a Poisson distribution, since they result from independent infection events (Paludan et al., J. Virol 63: 5201-7, 1989). Consequently, irrespective of the type of vector integrated as a provirus, none of the transcripts of the integrated retroviral sequence will be able to be mobilized or escape from the host cell.

### Example 12

### Homodimer Formation Based on Single Vector Systems

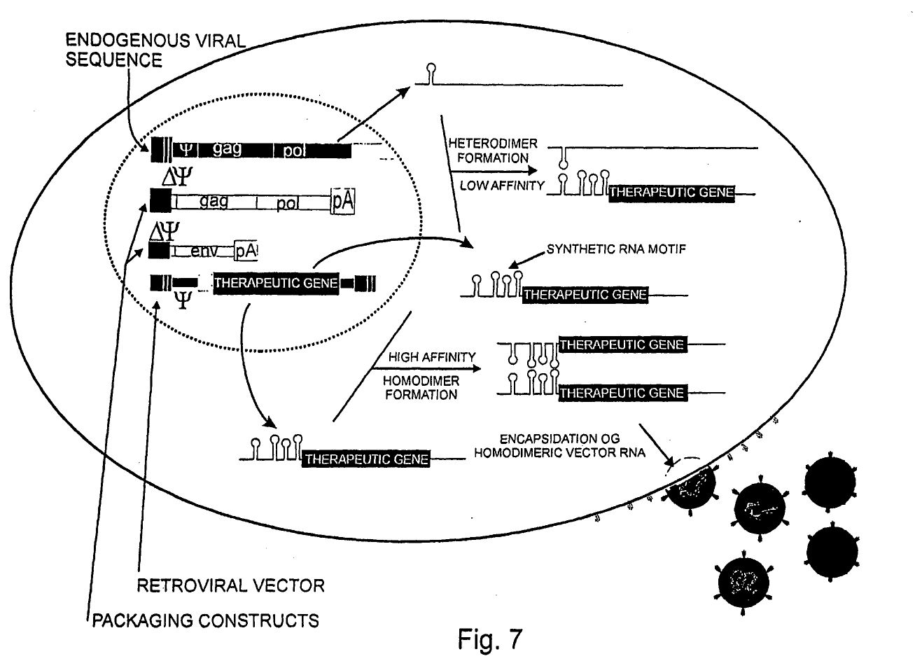

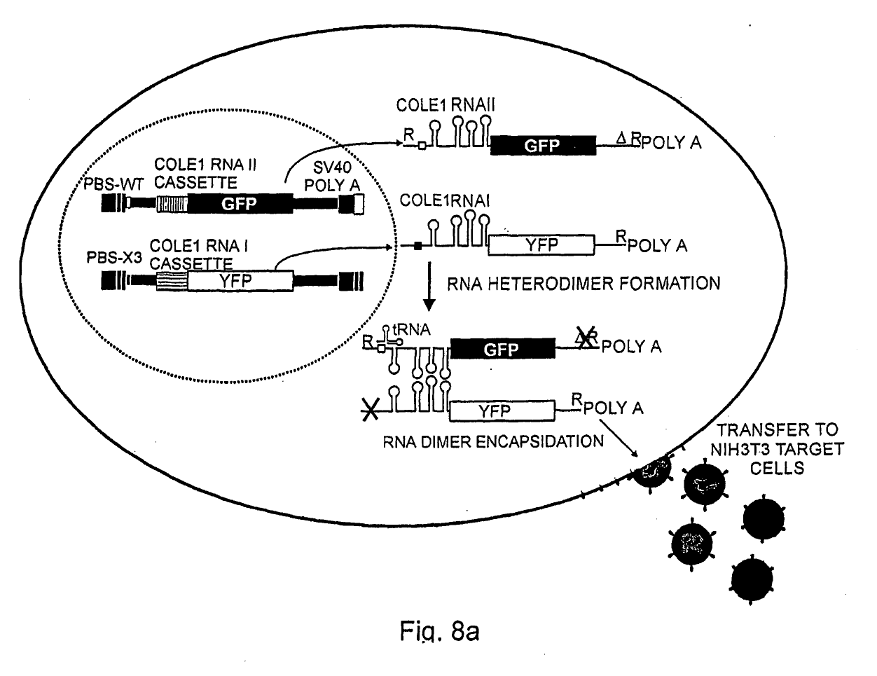

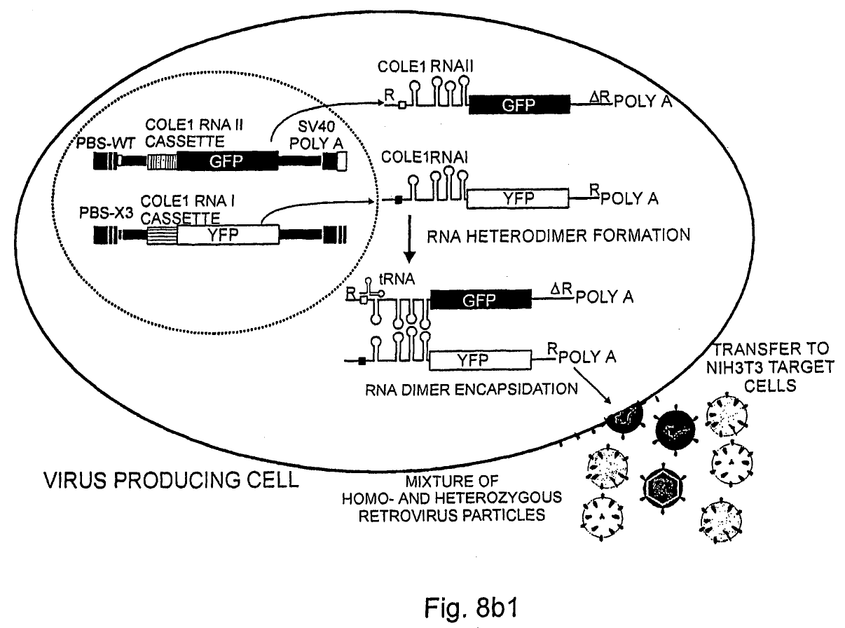

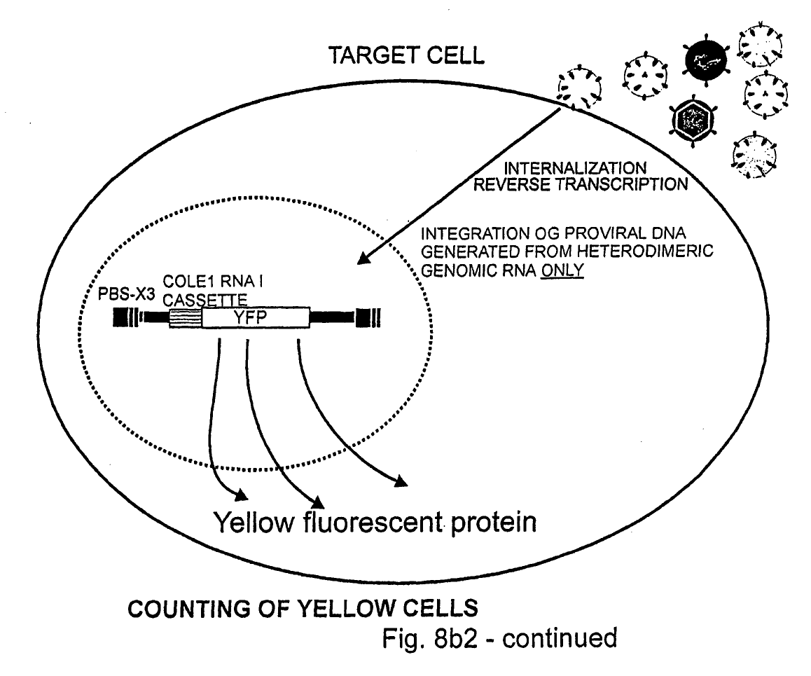

In the single vector system a vector for use in gene therapy and carrying a therapeutic gene of interest in introduced into a producer cell which contains two packaging constructs carrying the gag gene and the pol gene on a construct and the env gene on another construct. The cell further contains endogenous viral sequences (see FIG. 7). In order that the packaging within the cell selects for the homodimer produced by the gene therapeutic vector, said vector contains a synthetic RNA motif upstream of the therapeutic gene, which motif promotes the hybridisation between transcript from said vector. Since in this system, much more homodimers between transcripts from the gene therapeutic vector versus dimers involving transcripts from the packaging construct and the endogenous sequences, respectively, are formed, the virus particles formed mainly contain the desired dimer of the gene therapeutic vector sequences.

### Example 13

### Improving Safety of Two Vector Systems

The major difference to example 12 above resides in the fact that besides the gene therapeutic vector a further so-called “rescue vector” is used, the gene therapeutic vector containing the ColE1 RNAI sequence upstream of the therapeutic gene and the rescue vector the ColE1 RNAII sequence in a corresponding position. These two sequences promote the formation of a dimer between the rescue vector transcript and the retroviral vector transcript due to complimentary sequences within the Col E1 RNAI element and the Col E1 RNAII element. Again, also in this case, the dimer formation between the transcripts from the gene therapeutic vector and the rescue vector is favoured over any other dimer formation, which means that the likelihood of packaging the desired dimer is increased. The so produced viral particle is then introduced into the cells of the patient to be treated and the desired therapeutic gene is integrated into the target cell genome.

1. A retroviral vector system comprising at least one retroviral vector which has at least one modified heterologous or synthetic dimerisation sequence not present in its wild type state, said modification resulting in a reduction of the recombination frequency between a transcript of said vector that includes a transcript of the sequence modification, and at least one transcript of at least one different retrovirus present in a cell wherein the vector is present, said reduction being at least 2-fold relative to that of a corresponding transcript from the wild type retroviral vector.

2. A system according to claim 1 comprising a first and a second retroviral vector, the first vector lacking an initiation site for reverse transcription that is present in the second vector.

3. A system according to claim 1 in which the vector, relative to the wild type vector from which it is derived, has a transduction titer which is at least 25%.

4. A system according to claim 2 in which the two vectors, when present in a cell in a ratio in the range of 1:9 to 9:1, have a transduction titer which is at least 1% relative to the transduction titer of the wild type vector(s) from which they are derived.

5. A system according to any of claims 1 or 3 wherein the vector shows a reduction of the recombination frequency between the transcript of said vector and the at least one transcript of a different retrovirus present in a cell where the vector is present which is at least 5-fold.

6. A system according to any of claims 1-5 wherein the vector shows a reduction of recombination frequency between a transcript of said vector which includes the sequence modification, and a multiplicity of transcripts of one or more different retroviruses present in a cell where the vector is present.

7. A system according to any of claims 1-6 wherein the sequence modification in the vector is a substitution, deletion or addition of one or more bases.

8. A system according to any of claims 1-7 in wherein the sequence modification in the vector is a rearrangement or translocation of one or more bases in the sequence.

9. A system according to any of claims 1-8 wherein the modification of the vector is a substitution or an insertion of a dimerisation sequence of a different homologous or heterologous dimerisation sequence.

10. A system according to claim 9 wherein the dimerisation sequence is a palindromic or a kissing loop structure.

11. A system according to claim 7 where the modification in the vector is a substitution of a kissing loop structure with a least one kissing loop structure selected from the group of kissing loops sequences consisting of the kissing loop structure identified in MLV, ALV, HaSV, HIV-1, HIV-2, Coxsackie B virus and Porcine Arterivirus.

12. A system according to any of the preceding claims comprising a first and a second functionally and/or replicationally impaired retroviral vector in which the dimerisation sequences of the two vectors are different.

13. A system according to claim 12 in which the two different sequences are a pair selected from the group of sequence pairs consisting of sequences of plasmid origin: ColE1 (RNA I and RNA II), IncF (cop A RNA and repA mRNA), IncI (inc RNA and repZ mRNA), ColE2 (copRNA and repmRNA), R1162 (ct RNA and rep1 mRNA), R6K (silencer and activator), pT181 (RNA I and repC mRNA), IncF (finP RNA and traJ mRNA), IncFII (sok RNA and hok mRNA); of phage origin: lambda (aQ RNA and Q mRNA), lambda (oop RNA and cII mRNA), P22 (sar RNA and ant mRNA), P1 (c4 repressor and ant mRNA), P7 (c4 repressor and ant mRNA); of transposal origin: IS10 (RNA-OUT and tnp mRNA); and of bacterial origin: Escherichia coli (micF RNA and ompF mRNA) and E. coli (tic RNA and crp mRNA).

14. A cell, which has been transfected with at least one retroviral vector of a system according to any of claims 1-13.

15. A packaging cell for replication of at least one retroviral transfer vector of a system according to any of claims 1-13, where the cell is a mammalian or avian cell which has been transformed by the insertion of one or more DNA sequences carrying the information for the production of viral proteins required in trans for replication of said at least one retroviral transfer vector.

16. A virus particle containing at least one of the transcripts of the virus vector system according to any of claims 1-13.

17. A process for preparing a retroviral vector system, comprising the step of introducing at least one sequence modification in a dimerisation sequence, said modification resulting in a reduction of the recombination frequency between a transcript of said vector that includes a transcript of the sequence modification, and at least one transcript of at least one different retrovirus present in a cell wherein the vector is present, said reduction being at least 2-fold relative to that of a corresponding transcript from the wild type retroviral vector.

18. A method for improving the safety of a gene therapy vector system comprising the use of a retrovirus based vector system according to any of claims 1-13.

19. A method for determining the capability of a sequence pair comprising two identical or two different sequences to promote homodimer and/or heterodimer formation of transcripts derived from a retroviral vector system, the method comprising the step of introducing at least one retroviral vector of said vector system into a host cell and evaluating the frequency of homodimer and/or heterodimer formation of transcripts derived from said vector system by quantitating the relative number of specific recombination events that have occured, said number of specific recombination events being obtained by a method comprising the following steps;
a) selecting said sequence pair.
b) inserting one member of said sequence pair into one retrovirus vector containing a selectable marker gene and a non-functional primer binding site (PBS), and inserting the other member of said sequence pair into another retrovirus vector containing a functional primer binding site (PBS) but not containing the same selectable marker gene as the previous vector,
c) co-introduce the two retrovirus vectors of step (b) into a suitable pagaging cell which provides the necessary means to allow formation of infective retrovirus particles containing the information from both of said two retrovirus vectors of step (b),
d) recovering virus containing media and infect a culture of suitable host cells which do not contain the selectable marker gene of the said one retrovirus vector containing a non-functional primer binding site of step (b),
e) subject the transfected host cells of step (d) to a selection procedure which only allow cells that have been infected with virus particles containing said selectable marker gene of step (d) to form colonies, and
f) quantitate the number of specific recombination events from the number of resistant colonies obtained.

20. The use of a retroviral vector system according to any of claims 1-13 or a viral particle according to claim 16 for preparing a composition useful in gene therapy.

21. The use of a retroviral vector system according to any of claims 1-13 or a viral particle according to claim 16 for the manufacturing of a medicament for gene therapy.

22. The use of the cell according to claim 14 or 15 for the manufacturing of a medicament.

23. A pharmaceutical formulation comprising a vector system according to any of claims 1-13 and/or a viral particle according to claim 16.

24. A vaccine comprising a viral particle according to claim 16 or a part of said viral particle.


SEQUENCE LISTING (47 sequences)

SEQ ID NO: 1 - DNA, 35 bp, Artificial Sequence
  forward primer for amplification of Pac gene
atcgggggatcccttccatgaccgagtacaagccc

SEQ ID NO: 2 - DNA, 40 bp, Artificial Sequence
  reverse primer for amplification of Pac gene
tatccagatgaacagcattcgcgggtcgtggggcgggcgt

SEQ ID NO: 3 - DNA, 39 bp, Artificial Sequence
  forward  MLEV primer
agattgattgactgcccacctcgggggtctttcatttgg

SEQ ID NO: 4 - DNA, 29 bp, Artificial Sequence
  reverse  MLEV primer
gggcgcccctgcgctgacagccggaacac

SEQ ID NO: 5 - DNA, 45 bp, Artificial Sequence
  Akv U5 forward primer
gggaattctaccttacgtttccccgaccagagctgatgttctcag

SEQ ID NO: 6 - DNA, 28 bp, Artificial Sequence
  Akv  reverse primer
ggtgggcagtcaatcaatctgaggagac

SEQ ID NO: 7 - DNA, 20 bp, Artificial Sequence
  Overlap forward primer or product C ON5
gggaattctaccttacgttt

SEQ ID NO: 8 - DNA, 40 bp, Artificial Sequence
  Oligonucleotide ON1
ctgattctgtactagtatcgatactagatctgtatctggc

SEQ ID NO: 9 - DNA, 6 bp, Artificial Sequence
  the alternative palindrome KL-altpal
atcgat

SEQ ID NO: 10 - DNA, 37 bp, Artificial Sequence
  ON 8
caggtcgacggatccgtttttagaagcggtccaaaac

SEQ ID NO: 11 - DNA, 37 bp, Artificial Sequence
  ON 9
caggtcgacggatccgatctcgaaaacacttaaagac

SEQ ID NO: 12 - DNA, 40 bp, Artificial Sequence
  ON 4
ctgattctgtactagttaggatactagatctgtatctggc

SEQ ID NO: 13 - DNA, 6 bp, Artificial Sequence
  non-palindromic sequence
taggat

SEQ ID NO: 14 - DNA, 40 bp, Artificial Sequence
  ON 7
ctgattctgtactagtatcctaactagatctgtatctggc

SEQ ID NO: 15 - DNA, 6 bp, Artificial Sequence
  KL-(matchnonpal)
atccta

SEQ ID NO: 16 - DNA, 16 bp, Artificial Sequence
  Upstream primer
ccgtcgggaggtaagc

SEQ ID NO: 17 - DNA, 44 bp, Artificial Sequence
  downstream primer
cgtcggatcctactacgcctcgagtgcctctgagacgtctccca

SEQ ID NO: 18 - DNA, 57 bp, Artificial Sequence
  Upstream primer on a pPBSPRO244-ksi Akv-Neo template
actcgaggcgtagtaggatccgacgcagcggccgcacctacagccaagcttcacgct

SEQ ID NO: 19 - DNA, 50 bp, Artificial Sequence
  Fragment C and D Upstream primer
cgcgcgctcgagtgcaaacaaaaaaacaccgctaccaacggtggtttgtt

SEQ ID NO: 20 - DNA, 101 bp, Artificial Sequence
  Fragment C downstream primer
tgtaaggaaaaaagcggccgcactggtaaccggattagcagagcgatgatggcacaaacggtgctacagagttctgaagtagtggcccgactacggctaca

SEQ ID NO: 21 - DNA, 43 bp, Artificial Sequence
  Fragment D downstream primer
tgtaaggaaaaaagcggccgcacagtatttggttatctgcgct

SEQ ID NO: 22 - DNA, 36 bp, Artificial Sequence
  Fragment E upstream primer
gatgtgccgctcgagacagtatttggtatctgcgct

SEQ ID NO: 23 - DNA, 57 bp, Artificial Sequence
  Fragment E downstream primer
tgtaaggaaaaaagcggccgcaacaaaaaaaccaccgctaccaacggtggtttgttt

SEQ ID NO: 24 - DNA, 43 bp, Artificial Sequence
  pac gene primer containing NotI
aggatccgacgcagcggccgcaccatgaccgagtacaagccca

SEQ ID NO: 25 - DNA, 37 bp, Artificial Sequence
  pac gene primer containing BsmI restriction sites
tatccagatgaacagcattctcaggcaccgggcttgc

SEQ ID NO: 26 - DNA, 63 bp, Artificial Sequence
  primer 148760
agcttacctcccgacggtgggtcgcgatgtgtgtgtggcccgggcagtcaatcactctgagga

SEQ ID NO: 27 - DNA, 22 bp, Artificial Sequence
  primer A6600
cagcttgtctgtaagcggatgc

SEQ ID NO: 28 - DNA, 23 bp, Artificial Sequence
  primer 147654
gacccaccgtcgggaggtaagct

SEQ ID NO: 29 - DNA, 28 bp, Artificial Sequence
  primer 137599 or product A ON2
ggcgcccctgcgctgacagccggaacac

SEQ ID NO: 30 - DNA, 40 bp, Artificial Sequence
  oligonucleotide 150500
ctgcccagcctgggggtctttcattgggctcgtccgggat

SEQ ID NO: 31 - DNA, 69 bp, Artificial Sequence
  oligonucleotide 150501
cggtgggtcggtggtccctgggcgggggtctccaaatcccggacgagcccaatgaaagacccccaggct

SEQ ID NO: 32 - DNA, 10 bp, Artificial Sequence
  palindrome at position 209-218 in Akv/AkvB
gctggccagc

SEQ ID NO: 33 - DNA, 10 bp, Artificial Sequence
  Alternative palindrome at position 209-218 in Akv/AkvB
gcaccggtgc

SEQ ID NO: 34 - DNA, 21 bp, Artificial Sequence
  AkvB plasmid upstream primer A
gggtctgacgctcagtggaac

SEQ ID NO: 35 - DNA, 50 bp, Artificial Sequence
  AkvB plasmid downstream primer B
gacagagacggagacaaaacgatcgcaccggtgcttacctcccgacggtg

SEQ ID NO: 36 - DNA, 24 bp, Artificial Sequence
  AkvB plasmid downstream primer C
gatcgttttgtctccgtctctgtc

SEQ ID NO: 37 - DNA, 25 bp, Artificial Sequence
  AkvB plasmid downstream primer D
gcccccgatgcctctgagacgtctc

SEQ ID NO: 38 - DNA, 24 bp, Artificial Sequence
  Product A ON1
gcagacccctgcccagggaccacc

SEQ ID NO: 39 - DNA, 46 bp, Artificial Sequence
  Product B ON3
gggaattctaccttacgtttccccgaccagagctgaatgttcacag

SEQ ID NO: 40 - DNA, 24 bp, Artificial Sequence
  Product B ON4
ggtggtccctgggcaggggtctcc

SEQ ID NO: 41 - DNA, 22 bp, Artificial Sequence
  Product C ON6
cttcctttagcagcccttgcgc

SEQ ID NO: 42 - DNA, 30 bp, Artificial Sequence
  PBS-leader upstream primer U3
gcggccgcgattcccagatgaccggggatc

SEQ ID NO: 43 - RNA, 22 nt, retrovirus  PBSPro476 psiAkv-neo
  misc_RNA (0)...(0): kissing loop sequence of PBSPro476 psiAkv-neo
guacuaguuagcuaacuagauc

SEQ ID NO: 44 - RNA, 22 nt, retrovirus
  misc_RNA (0)...(0): kissing loop sequence of PBSProKLaltpal Akv-neo and of MLEVleaderKLaltpal Akv-pac
guacuaguaucgauacuagauc

SEQ ID NO: 45 - RNA, 22 nt, retrovirus
  misc_RNA (0)...(0): kissing loop sequence of PBSProKL-nonpal Akv-neo and of MLEVleaderKLnonpal Akv-pac
guacuaguuaggauacuagauc

SEQ ID NO: 46 - RNA, 22 nt, retrovirus
  misc_RNA (0)...(0): kissing loop sequence of MLEVleader Akv-pac
guacuaguuggcuaacuagauc

SEQ ID NO: 47 - RNA, 22 nt, retrovirus
  misc_RNA (0)...(0): kissing loop sequence of MLEVleaderKLmatchnonpal Akv-pac
guacuaguauccuaacuagauc